(19) United States
(12) Patent Application Publication
(10) Pub. No.: US 2024/0166572 A1
(43) Pub. Date: May 23, 2024

(54) Holistic and Environmentally-Friendly Systems for Crop, Soil, Water and Livestock Management

(71) Applicant: Locus Solutions IPCo, LLC, Solon, OH (US)

(72) Inventors: Paul ZORNER, Encinitas, CA (US); Sean FARMER, Ft. Lauderdale, FL (US)

(21) Appl. No.: 17/771,638

(22) PCT Filed: Mar. 18, 2022
(86) PCT No.: PCT/US2022/020877

§ 371 Date: Sep. 29, 2022

Related U.S. Application Data
(60) Provisional application No. 63/163,819, filed on Mar. 20, 2021.
(60) Provisional application No. 63/235,360, filed on Aug. 20, 2021.

(51) Int. Cl. C05F 11/08; C05G 1/00; C05G 3/80; C05G 5/12

(52) U.S. Cl. CPC C05F 11/08; C05G 1/00; C05G 3/80; C05G 5/12

(57) ABSTRACT

The subject invention provides environmentally-friendly systems for the management of crops, soil, water and livestock. More specifically, the invention provides for a holistic approach using microbe-based and/or biosurfactant-based soil treatment compositions to address the various pain points experienced by growers and livestock producers, including those involving soil and water management.

### CROSS-REFERENCE TO RELATED APPLICATIONS

This application claims priority to U.S. Provisional Patent Application Nos. 63/163,819, filed Mar. 20, 2021, and 63/235,360, filed Aug. 20, 2021, both of which are incorporated herein by reference in their entireties.

### BACKGROUND OF THE INVENTION

Agriculture and livestock production are essential industrial operations for feeding the world's population and producing other valuable commodities. The production of crops is vital for producing foods, pharmaceuticals, textiles and structural materials, as well as feed for producing livestock. Livestock consume feed and are important for the production of meat, dairy, textile and medicinal products, while also producing manure to help fertilize crops. These deeply interrelated industries, however, can experience hardships, costs and unpredictable yields based on the climate and various environmental and man-made influences.

For example, degradation of soil is a growing problem, particular for high organic content soils such as muck soils. Certain types of soils can degrade over time (sometimes referred to as “subsidence”) as a result of draining, which leads to oxygenation of the soil environment and speeds up the aerobic breakdown of organic matter by soil microorganisms and/or their extracellular enzymes. Furthermore, dry surface soil can be eroded by wind.

Additionally, as the amount of microbial decomposition of carbon-rich organic matter in soil increases, the result is an increase in the rate of atmospheric greenhouse gas (GHG) emissions, such as carbon dioxide, methane and nitrous oxide, from these processes and a decrease in the amount of soil organic carbon (SOC).

Soil organic carbon (SOC) is an important component of soil matter and consists mainly of plant and animal tissue remains, live microbial biomass, and the by-products of microbial processes, as well as organo-mineral complexes. As part of the broader carbon exchange cycle, even minor changes in SOC can have a large impact on the levels of atmospheric carbon dioxide in a region (1 Pg of soil carbon stock=0.47 ppm of atmospheric CO2). Sequestration of SOC occurs when CO2 is transferred from the atmosphere into the soil by way of plant residues and other organic materials, which are stored in the soil with a long mean residence time (MRT). SOC sequestration can be achieved by, for example, increasing plant growth, retaining above and below-ground plant biomass, and/or protecting and stabilizing the SOC against erosion and decomposition.

A positive soil carbon budget is created by increasing the input of biomass carbon to exceed the SOC losses by erosion and decomposition. The rate of decomposition of biomass is affected by many factors, including, for example, climate, moisture levels, and types of plant matter—live or dead—present in the soils (Lal 2017).

An additional important factor impacting the rate of soil carbon accumulation is soil aggregate formation and stability. Healthy and robust root systems are effective for forming and stabilizing carbon-capturing soil aggregates, where organic matter and minerals become enmeshed in the roots. Soil microorganisms (e.g., fungal hyphae), and the growth by-products thereof (e.g., polysaccharides), can also facilitate association of carbon with soil mineral particles to form and stabilize these aggregates. Furthermore, studies have shown that the greater the soil aggregate size, the lower the degradation of soil by extracellular enzymes produced by microorganisms that consume organic matter in soil (Trivedi, P. et al. 2017; Trivedi, P. et al. 2015; Possinger et al. 2020; Grandy 2007).

Livestock production also has its pain points. While many analyses focus on the GHG emissions produced via ruminal digestion, feed and bedding are also sources of GHG. Almost 60% of the global biomass harvested worldwide (e.g., corn and other forage) enters the livestock subsystem as feed or bedding material. Soil carbon dioxide emissions are produced as a result of soil carbon dynamics (e.g., decomposing plant residues, mineralization of soil organic matter, land use change, etc.), the manufacturing of synthetic fertilizers and pesticides, and from fossil fuel use in on-farm agricultural operations.

With fertilizer use in particular, nitrous oxide is emitted when organic and inorganic fertilizers are applied to the soil. These fertilizers are susceptible to loss by leaching and denitrification before crop uptake. Over-dependence and long-term use of certain chemical fertilizers, pesticides and antibiotics can alter soil ecosystems, reduce stress tolerance, increase the prevalence of resistant pests, and impede plant growth and vitality.

One further issue that can have drastic effects on both the agriculture and livestock industries is water usage. In certain areas of the country, over farming, over-industrialization and/or over-developing are leading to a reduction in ground water and aquifer levels. In other areas, droughts can occur, leading to widespread water shortages and reduced crop yields. The amount of water required to irrigate large tracts of farmland, as well as the amount of water needed for drinking by livestock animals, necessitates increased water use efficiency for both industries, where less water is required to achieve a desired production level.

The economic costs and environmental impacts of current methods of crop and livestock production continue to burden the sustainability of the agriculture and livestock industries. It can be difficult for a grower to detect, address and monitor the various paint points while also maximizing yields and revenue for a growing season. Thus, there is a continuing need for improved systems that address the needs of growers and herders with efficiency and sustainability.

### BRIEF SUMMARY OF THE INVENTION

The subject invention provides environmentally-friendly systems for the management of crops, soil, water and/or livestock. More specifically, the invention provides for a holistic approach using soil treatment compositions to address the various pain points involved with crop and livestock production, including soil and water management. This holistic approach includes one or more of, for example:
(a) Enhancing plant health, growth and/or yields, including livestock feed crops;
(b) Reducing fertilizer usage;
(c) Reducing the use of chemical or petroleum-based surfactants applied as wetting agents, dispersants, antifoamers, and other additives in irrigation, herbicides, pesticides and fertilizers;
(d) Enhancing soil health though rebuilding of degraded soils, preventing degradation of soils, and/or improving soil aggregate formation;
(e) Improving dispersion of water, nutrients and salts in soil;
(f) Recharging depleted aquifers;
(g) Improving crop and livestock water use efficiency;
(h) Reducing atmospheric greenhouse gas emissions resulting from agricultural practices and livestock feed production, including manure management;
(i) Enhancing sequestration of carbon in soil, vegetation and microbial biomass; and/or
(j) Reducing the carbon footprint and/or carbon intensity of producing crops and livestock, which can also result in a reduction in the number of carbon credits used by a farmer or livestock producer.

In preferred embodiments, the subject invention provides microbe-based soil treatment compositions, as well as methods that utilize these products. In certain embodiments, the invention utilizes microbial growth by-products, such as, for example, biosurfactants. Advantageously, in preferred embodiments the subject invention utilizes organic, non-GMO components.

In certain embodiments, the soil treatment composition comprises one or more beneficial microorganisms. In preferred embodiments, the beneficial microorganisms are non-pathogenic, soil-colonizing fungi, yeasts and/or bacteria capable of producing one or more of the following: surface active agents, such as lipopeptides and/or glycolipids; bioactive compounds with antimicrobial and immune-modulating effects; polyketides; acids; peptides; anti-inflammatory compounds; enzymes, such as proteases, amylases, and/or lipases; and sources of amino acids, vitamins, and other nutrients.

In preferred embodiments, the microorganism is a non-pathogenic bacterium, yeast and/or fungus selected from, for example, Trichoderma spp., Bacillus spp., Wickerhamomyces anomalus, Myxococcus xanthus, Pseudomonas chlororaphis, Starmerella bombicola, Saccharomyces boulardii, Pichia occidentalis, Pichia kudriavzevii, Meyerozyma guilliermondii, Meyerozyma caribbica, mycorrhizal fungi, nitrogen fixers (e.g., Azotobacter vinelandii) and/or potassium mobilizers (e.g., Frateuria aurantia).

The species and ratio of microorganisms and other ingredients in the composition can be determined according to, for example, the geographic region where treatment will occur, environmental factors such as drought and/or flooding, the species of livestock animal(s) that may consume the plants and/or harvested products thereof, the health status of the farmland at the time of treatment, as well as other factors. Thus, the composition can be customized for any given location.

In certain exemplary embodiments, the soil treatment composition comprises a first microorganism and a second microorganism, growth by-products thereof, and, optionally, one or more sources of nutrients. In a specific exemplary embodiment, the first microorganism is Trichoderma harzianum and the second microorganism is Bacillus amyloliquefaciens (e.g., B. amyloliquefaciens NRRL B-67928).

In certain exemplary embodiments, the soil treatment composition comprises a yeast such as, for example, Wickerhamomyces anomalus (e.g., NRRL Y-68030) or Meyerozyma caribbica (e.g., M. caribbica MEC14XN).

In certain exemplary embodiments, the soil treatment composition comprises microbial growth by-products, which can include, for example, the fermentation medium in which the microbes were cultivated, and/or any leftover nutrients from cultivation. The cells may remain in the medium, removed entirely from the medium, and/or removed to a point where only residual cellular matter remains in the medium. The growth by-products can also comprise metabolites or other biochemicals produced as a result of cell growth, including, for example, biosurfactants. In some embodiments, soil treatment composition comprises the growth by-product after it has been extracted from and/or purified from the fermentation medium.

In one embodiment, the microbe-based soil treatment composition is applied to a tract of land, such as farmland, pastureland, forest land, or tracts cleared by natural fire and prescribed burns, wherein the soil treatment composition provides one or more direct or indirect benefits to the plants and/or soil of the land, which contribute to the holistic approach for improving crop and livestock production.

These benefits include, for example, promoting increased above- and below-ground plant biomass per plant, increased high-carbon content polymers in plant tissue, increased numbers of plants per unit of area, increased uptake by microorganisms of organic compounds secreted by plants, reduced fertilizer usage, improved nutrient solubilization, increased size and/or quantity of carbon- and water-binding soil-mineral aggregates, improved retention and dispersion of water and/or nutrients in soil, reduced pooling of water and evaporation from soil, increased water penetration into deeper soil and aquifers, reduced soil salinity and pollutant content, and increased microbial biomass and necromass in soil.

In one embodiment, the microbe-based soil treatment composition is applied to a lagoon or livestock manure pond, wherein the soil treatment composition can increase the rate of waste decomposition, improve the separation and dewatering of manure, and control methanogenic microorganisms present in the manure. Accordingly, the methods can contribute to reduced GHG emissions from manure and provide for efficient treatment, processing and recycling of manure as crop fertilizers.

In one embodiment, the microbe-based soil treatment composition is applied to soil or land experiencing drought and/or aquifer depletion, wherein the soil treatment composition increases the wettability of soil, improves retention and dispersion of water in soil, improves the drainage and dispersion of pooling water in hydrophobic soils, reduces water loss due to evaporation, and/or increases water penetration into deeper soil layers and groundwater sources, such as aquifers. Accordingly, the methods can contribute to improved water use efficiency, improved drought management, and recharging of depleted aquifers.

In certain embodiments, the subject methods can further comprise applying materials to enhance microbe, plant, and/or soil health during application (e.g., adding germination enhancers, prebiotics, nutrients and/or mineral sources). In one embodiment, these additional materials can include, for example, sources of magnesium, phosphate, nitrogen, potassium, selenium, calcium, sulfur, iron, copper, zinc, proteins, vitamins and/or carbon; prebiotics, such as, for example, one or more of biochar, kelp extract, fulvic acid, chitin, humate and humic acid; and/or rock dust.

The methods and compositions of the subject invention can be used either alone or in combination with additional components, such as herbicides, fertilizers, pesticides and/or other soil amendments. Preferably, the additional components are non-toxic and environmentally-friendly. The exact materials and the quantities thereof can be determined by, for example, a grower or soil scientist having the benefit of the subject disclosure.

The methods of the subject invention can utilize standard methods and equipment that are used for maintenance of farmland. For example, the soil treatment composition can be applied in liquid form using an irrigation system. The composition can also be applied as a granule, as a coating, or impregnated into prills. Additionally, the composition can be applied using a manual spreader, such as a broadcast spreader, a drop spreader, a handheld spreader, or a handheld sprayer.

In some embodiments, the systems of the subject invention further involve the monitoring of various inputs and outputs of crop and livestock production. For example, following application of a soil treatment composition to a tract of land, factors such as water usage, soil moisture content and dispersion, fertilizer usage, soil nutrient content and dispersion, soil microbial populations, soil organic content, generation of GHG emissions, fossil fuel usage, plant growth, health and yields, and even the presence of pests, can be monitored. Accordingly, the system can be adjusted throughout implementation to account for changes in these factors and make appropriate adjustments to the inputs in real time.

Monitoring can be performed in place and/or via wireless methods using known methods in the art. For example, in some embodiments, fixed location sensors can be used to detect GHG emissions from a field and transmit data to a network for remote monitoring. Nanocrystal detection assays, such as those described in U.S. Patent Application Publication No. US-2020-0102602-A1, incorporated herein by reference, can be used for monitoring microbial populations and other analytes in soil.

Advantageously, the holistic approaches can be centralized and performed by a single entity, which performs and/or facilitates production, formulation/customization, transportation and application of the soil treatment compositions, as well as the monitoring of the factors listed above. The central entity can utilize data collected from a single customer and/or a collection of customers to formulate future courses of action actions based on observed developments and trends.

In some embodiments, the central entity can serve as a general contractor, with sub-contractors performing one or more of the production, formulation/customization, transportation and application of the soil treatment compositions, as well as monitoring.

Advantageously, the systems of the subject invention can increase the efficiency and reduce the financial and environmental costs of agriculture and livestock practices. In particular, the compositions and methods utilized according to the subject invention can help in preserving valuable natural resources, such as soil and water, while improving production of valuable plant and animal-based commodities.

### BRIEF DESCRIPTION OF THE FIGURES

FIG. 1 shows yield increase for corn treated with a soil treatment composition according to an embodiment of the subject invention. Treatments were applied alongside varying concentrations of fertilizer and compared with grower's practice fertilization (control).

FIG. 2 shows corn yield increase comparison between two different soil treatment compositions according to embodiments of the subject invention.

FIG. 3 shows soybean yield increase comparison between two different soil treatment compositions according to embodiments of the subject invention.

FIG. 4 depicts the increase in soil carbon sequestration observed for corn and soybean crops treated with soil treatment compositions according to embodiments of the subject invention over control crops.

FIG. 5 shows nitrous oxide emissions measured over at least a 43 day period from corn fields treated with a urea slurry and a soil treatment composition according to an embodiment of the subject invention (Treatment 1), compared with the urea slurry alone (Treatment 2) and an untreated control (Treatment 3). The black arrows indicate Day 3 and Day 23, marking the points at which the soil treatment composition was applied.

### DETAILED DESCRIPTION OF THE INVENTION

The subject invention provides environmentally-friendly systems for the management of crops, soil, water and livestock. More specifically, the invention provides for a holistic approach using soil treatment compositions to address the various pain points experienced by growers and livestock producers, including those involving soil and water management.

This holistic approach provides solutions to problems such as, for example, erosion and degradation of soil; reduced crop health, growth and yields; over-fertilization and fertilizer runoff; low water use efficiency; persistence of chemical or petroleum-based surfactants in soil; poor nutrient solubilization and bioavailability; and the growing carbon footprint/carbon intensity of crop and livestock production due to, for example, GHG emissions from fossil fuel-burning equipment, microbial decomposition of soil, fertilizer usage, livestock digestion, and livestock manure.

### Selected Definitions

As used herein, “agriculture” means the cultivation and breeding of plants for food, fiber, biofuel, medicines, cosmetics, supplements, ornamental purposes and other uses. According to the subject invention, agriculture can also include horticulture, landscaping, gardening, plant conservation, forestry and reforestation, pasture and prairie restoration, orcharding, arboriculture, and agronomy. Further included in agriculture are the care, monitoring and maintenance of soil.

As used herein, a “broth,” “culture broth,” or “fermentation broth” refers to a culture medium comprising at least nutrients and microorganism cells.

As used herein, the term “carbon use efficiency” or “CUE” refers to a generalized measure of the efficiency by which microbes allocate carbon taken up towards growth and biomass production versus respiration. CUE can be calculated as growth (biomass production) over the sum of CO₂ production/emissions and growth. Microorganisms are often categorized as “low CUE” or “high CUE,” where a CUE greater than 0.50 is considered high, and a CUE lower than 0.50 is considered low.

Unless the context requires otherwise, the phrases “fermenting,” “fermentation process” or “fermentation reaction” and the like, as used herein, are intended to encompass both the growth phase and product biosynthesis phase of the process.

As used herein, “farmland” includes any tract of land in which plants are grown, cultivated and/or managed for human interests. Farmland includes:
pastures, or land containing mostly grasses, legumes and non-grass herbaceous plants, that is grazed by livestock;
meadows, which are typically ungrazed tracts of land that may be used for harvesting hay or other animal fodder,
rangelands, which include untended and human-tended grasslands, shrublands, woodlands, wetlands and deserts that are grazed by domestic livestock or wild animals; and
agricultural crops.

As used herein, “fodder” means any plant material that is harvested or otherwise cut for feeding livestock animals. Fodder can include, but is not limited to, grasses, forbs, shrubs, alfalfa, hay, straw, legumes, nuts, seeds, fruits, vegetables and/or crop residue.

As used herein, “forage” means any plant material that is growing in a tract of farmland and that is consumed by, or at least edible to, a livestock animal.

As used herein, “grain-fed” livestock means the livestock animals consume grains as part of their regular diet throughout the course of their life. The grains can comprise, for example, at least 10%, at least 25%, at least 50%, at least 75%, at least 85%, at least 95% or 100% of the animals' total feed supply. Grains include, but are not limited to, corn, oats, barley, wheat, sorghum, milo and soy.

In some embodiments, the grain-fed livestock is “grain-finished,” meaning the livestock animals spend the majority of their lives grazing and/or eating grasses and forage-based feeds, but then spend the last 4 to 6 months, for example, eating a predominantly grain-based diet (e.g., more than 50%, 60%, 75%, or 90% of caloric intake). This grain-based diet often comprises high-energy grains, such as corn, wheat and milo; however, in some instances, the animals also consume other diverse feed sources in addition to the feedlot grains, such as potato hulls, sugar beets, and hay.

As used herein, “grass-fed” livestock means the livestock animal eats exclusively grasses and forage throughout its entire life, starting after weaning.

As used herein, an “isolated” or “purified” compound is substantially free of other compounds, such as cellular material, with which it is associated in nature. A purified or isolated polynucleotide (ribonucleic acid (RNA) or deoxyribonucleic acid (DNA)) is free of the genes or sequences that flank it in its naturally-occurring state. A purified or isolated polypeptide is free of the amino acids or sequences that flank it in its naturally-occurring state. “Isolated” in the context of a microbial strain means that the strain is removed from the environment in which it exists in nature. Thus, the isolated strain may exist as, for example, a biologically pure culture, or as spores (or other forms of the strain) in association with a carrier.

As used herein, a “biologically pure culture” is a culture that has been isolated from materials with which it is associated in nature. In a preferred embodiment, the culture has been isolated from all other living cells. In further preferred embodiments, the biologically pure culture has advantageous characteristics compared to a culture of the same microbe as it exists in nature. The advantageous characteristics can be, for example, enhanced production of one or more growth by-products.

In certain embodiments, purified compounds are at least 60% by weight the compound of interest. Preferably, the preparation is at least 75%, more preferably at least 90%, and most preferably at least 99%, by weight the compound of interest. For example, a purified compound is one that is at least 85%, 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

As used herein, “enhancing” means improving or increasing. For example, enhanced plant health can mean improving the plant's ability grow and thrive, which can include increased seed germination and/or emergence, improved immune response against pests and/or diseases, and improved ability to survive environmental stressors, such as droughts and/or overwatering. Enhanced plant growth and/or enhanced plant biomass can mean increasing the size and/or mass of a plant above and/or below the ground (e.g., increased canopy/foliar volume, height, trunk caliper, branch length, shoot length, protein content, root size/density and/or overall growth index), and/or improving the ability of the plant to reach a desired size and/or mass. Enhanced yields can mean improving the end products produced by the plants in a crop, for example, by increasing the number, size and/or amount of fruits, leaves, roots, extracts and/or tubers per plant, and/or improving the quality of the fruits, leaves, roots extracts and/or tubers (e.g., improving taste, texture, brix, chlorophyll content and/or color).

“Livestock” animals, as used herein, are “domesticated” animals, meaning species that have been influenced, bred, tamed, and/or controlled over a sustained number of generations by humans, such that a mutualistic relationship exists between the animal and the human. Particularly, livestock animals include animals raised in an agricultural or industrial setting to produce commodities such as food, fiber and labor. Types of animals included in the term livestock can include, but are not limited to, alpacas, llamas, pigs (swine), horses, mules, asses, camels, dogs, ruminants, chickens, turkeys, ducks, geese, guinea fowl, and squabs.

In certain embodiments, the livestock animals are “ruminants,” or mammals that utilize a compartmentalized stomach suited for fermenting plant-based foods prior to digestion with the help of a specialized gut microbiome. Ruminants include, for example, bovines, sheep, goats, ibex, giraffes, deer, elk, moose, caribou, reindeer, antelope, gazelle, impala, wildebeest, and some kangaroos.

In specific exemplary embodiments, the livestock animals are bovine animals, which are ruminant animals belonging to the subfamily Bovinae, of the family Bovidae. Bovine animals can include domesticated and/or wild species. Specific examples include, but are not limited to, water buffalo, anoa, tamaraw, auroch, banteng, guar, gayal, yak, kouprey, domestic meat and dairy cattle (e.g., Bos laurus, Bos indicus), ox, bullock, zebu, saola, bison, buffalo, wisent, bongo, kudu, kewwel, imbabala, kudu, nyala, sitatunga, and eland.

A “metabolite” refers to any substance produced by metabolism (e.g., a growth by-product) or a substance necessary for taking part in a particular metabolic process. A metabolite can be an organic compound that is a starting material, an intermediate in, or an end product of metabolism. Examples of metabolites include, but are not limited to, biosurfactants, biopolymers, enzymes, acids, solvents, alcohols, proteins, vitamins, minerals, microelements, and amino acids.

The subject invention utilizes “microbe-based compositions,” meaning a composition that comprises components that were produced as the result of the growth of microorganisms or other cell cultures. Thus, the microbe-based composition may comprise the microbes themselves and/or by-products of microbial growth. The microbes may be in a vegetative state, in spore or conidia form, in hyphae form, in any other form of propagule, or a mixture of these. The microbes may be planktonic or in a biofilm form, or a mixture of both. The by-products of growth may be, for example, metabolites, cell membrane components, proteins, and/or other cellular components. The microbes may be intact or lysed. In preferred embodiments, the microbes are present, with growth medium in which they were grown, in the microbe-based composition. The microbes may be present at, for example, a concentration of at least 1×10⁴, 1×10⁵, 1×10⁶, 1×10⁷, 1×10⁸, 1×10⁹, 1×10¹⁰, 1×10¹¹, 1×10¹² or 1×10¹³ or more CFU per gram or per ml of the composition.

The subject invention further provides “microbe-based products,” which are products that are to be applied in practice to achieve a desired result. The microbe-based product can be simply a microbe-based composition harvested from a microbe cultivation process. Alternatively, the microbe-based product may comprise further ingredients that have been added. These additional ingredients can include, for example, stabilizers, buffers, appropriate carriers, such as water, salt solutions, or any other appropriate carrier, added nutrients to support further microbial growth, non-nutrient growth enhancers and/or agents that facilitate tracking of the microbes and/or the composition in the environment to which it is applied. The microbe-based product may also comprise mixtures of microbe-based compositions. The microbe-based product may also comprise one or more components of a microbe-based composition that have been processed in some way such as, but not limited to, filtering, centrifugation, lysing, drying, purification and the like.

As used herein “preventing” or “prevention” of a situation or occurrence means delaying, inhibiting, suppressing, forestalling, and/or minimizing the onset, extensiveness or progression of the situation or occurrence. Prevention can include, but does not require, indefinite, absolute or complete prevention, meaning it may still develop at a later time. Prevention can include reducing the severity of the onset of such a situation or occurrence, and/or stalling its development to a more severe or extensive situation or occurrence.

Ranges provided herein are understood to be shorthand for all of the values within the range. For example, a range of 1 to 20 is understood to include any number, combination of numbers, or sub-range from the group consisting of 1, 2, 3, 4, 5, 6, 7, 8, 9, 10, 11, 12, 13, 14, 15, 16, 17, 18, 19, 20, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein, “reduction” refers to a negative alteration, and the term “increase” refers to a positive alteration, wherein the negative or positive alteration is at least 0.25%, 0.5%, 1%, 5%, 10%, 15%, 20%, 25%, 30%, 35%, 40%, 45%, 50%, 55%, 60%, 70%, 75%, 80%, 85%, 90%, 95%, or 100%.

As used herein, “reference” refers to a standard or control condition.

As used herein, a “soil amendment” or a “soil conditioner” is any compound, material, or combination of compounds or materials that are added into soil to enhance the properties of the soil and/or rhizosphere. Soil amendments can include organic and inorganic matter, and can further include, for example, fertilizers, pesticides and/or herbicides. Nutrient-rich, well-draining soil is essential for the growth and health of plants, and thus, soil amendments can be used for enhancing the plant biomass by altering the nutrient and moisture content of soil. Soil amendments can also be used for improving many different qualities of soil, including but not limited to, soil structure (e.g., preventing compaction); improving the nutrient concentration and storage capabilities; improving water retention in dry soils; and improving drainage in waterlogged soils.

As used herein, “surfactant” refers to a compound that lowers the surface tension (or interfacial tension) between phases. Surfactants act as, e.g., detergents, wetting agents, emulsifiers, foaming agents, and dispersants. A “biosurfactant” is a surfactant produced by a living organism and/or using naturally-derived substrates.

As used herein, “water use efficiency,” or “WUE,” refers to the ratio of yields and/or biomass produced per unit of water applied. According to the subject invention, WUE can refer to the measure of plant yields/biomass, as well as animal yields/biomass (e.g., carcass weight) produced per unit of water applied

The transitional term “comprising,” which is synonymous with “including,” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates other embodiments that “consist” or “consist essentially” of the recited component(s).

Unless specifically stated or obvious from context, as used herein, the term “or” is understood to be inclusive. Unless specifically stated or obvious from context, as used herein, the terms “a,” “and” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof. All references cited herein are hereby incorporated by reference in their entirety.

### Holistic Systems for Improving Agriculture and Livestock Production

The subject invention provides environmentally-friendly systems for the management of crops, soil, water and livestock. More specifically, the invention provides for a holistic approach using microbial soil treatment compositions to address the various pain points involved with crop and livestock production, including soil and water management. This holistic approach includes one or more of, for example:
(a) Enhancing soil health though rebuilding of degraded soils, preventing degradation of soils, and/or improving soil aggregate formation;
(b) Improving dispersion of water, nutrients and salts in soil;
(c) Reducing chemical or petroleum-based surfactant usage;
(d) Enhancing plant health, growth and/or yields, including livestock feed crops;
(e) Reducing fertilizer usage;
(f) Recharging depleted aquifers;
(g) Improving crop and livestock water use efficiency;
(h) Reducing atmospheric greenhouse gas emissions resulting from agricultural practices and livestock feed production, including manure management;
(i) Enhancing sequestration of carbon in soil, vegetation and microbial biomass; and/or
(j) Reducing the carbon footprint and/or carbon intensity of producing crops and livestock, which can also result in a reduction in the number of carbon credits used by a farmer or livestock producer.

The subject invention can be used to achieve any one or a combination of any number of the above-listed goals. In some embodiments, some of the above goals over-lap one another such that achieving one, e.g., goal (b), will also help in achieving another, e.g., goal (e). In preferred embodiments, the subject invention provides microbe-based soil treatment compositions, as well as methods that utilize these products. Advantageously, the subject invention utilizes organic, non-GMO components.

In one embodiment, a unit tract of land, such as an acre or any other unit, is monitored to evaluate the attainment of the goal(s). Preferably, the monitoring is quantitative. In one embodiment, carbon credits are earned by, or in addition to, attainment of the goal(s).

In certain embodiments, the soil treatment composition comprises one or more beneficial microorganisms. In preferred embodiments, the beneficial microorganisms are non-pathogenic, soil-colonizing fungi, yeasts and/or bacteria capable of producing one or more of the following: surface active agents, such as lipopeptides and/or glycolipids; bioactive compounds with antimicrobial and immune-modulating effects; polyketides; acids; peptides; anti-inflammatory compounds; enzymes, such as proteases, amylases, and/or lipases; and sources of amino acids, vitamins, and other nutrients.

In preferred embodiments, the microorganism is a non-pathogenic bacterium, yeast and/or fungus selected from, for example, Trichoderma spp., Bacillus spp., Wickerhamomyces anomalus, Myxococcus xanthus, Pseudomonas chlororaphis, Starmerella bombicola, Saccharomyces boulardii, Pichia occidentalis, Pichia kudriavzevii, Meyerozyma guilliermondii, Meyerozyma caribbica, mycorrhizal fungi, nitrogen fixers (e.g., Azotobacter vinelandii) and/or potassium mobilizers (e.g., Frateuria aurantia).

The species and ratio of microorganisms and other ingredients in the composition can be determined according to, for example, the geographic region where treatment will occur, environmental factors such as drought and/or flooding, the species of livestock animal(s) that may consume the plants and/or harvested products thereof, the health status of the farmland at the time of treatment, as well as other factors. Thus, the composition can be customized for any given location.

In certain exemplary embodiments, the soil treatment composition comprises a first microorganism and a second microorganism, growth by-products thereof, and, optionally, one or more sources of nutrients. In a specific exemplary embodiment, the first microorganism is Trichoderma harzianum and the second microorganism is Bacillus amyloliquefaciens “B. amy” (e.g., B. amyloliquefaciens NRRL B-67928).

In one exemplary embodiment, the soil treatment composition comprises B. subtilis B4 (NRRL B-68031).

In one exemplary embodiment, the soil treatment composition comprises B. amy and B4.

In one exemplary embodiment, the soil treatment composition comprises a yeast such as, for example, Wickerhamomyces anomalus (e.g., NRRL Y-68030), Meyerozyma guilliermondii and/or Meyerozyma caribbica (e.g., M. caribbica “MEC14XN”).

In certain exemplary embodiments, the soil treatment composition comprises microbial growth by-products, which can include, for example, the fermentation medium in which the microbes were cultivated, and/or any leftover nutrients from cultivation. The cells may remain in the medium, removed entirely from the medium, and/or removed to a point where only residual cellular matter remains in the medium. The growth by-products can also comprise metabolites or other biochemicals produced as a result of cell growth, including, for example, biosurfactants. In some embodiments, soil treatment composition comprises the growth by-product after it has been extracted from and/or purified from the fermentation medium.

In one embodiment, the microbe-based soil treatment composition is applied to a tract of land, such as farmland, pastureland, forest land, or tracts cleared by natural fire and prescribed burns, wherein the soil treatment composition provides one or more direct or indirect benefits to the plants and/or soil of the land, which contribute to the holistic approach of improving crop and livestock production.

These benefits include, for example, promoting increased above- and below-ground plant biomass per plant, increased high-carbon content polymers in plant tissue, increased numbers of plants per unit of area, increased uptake by microorganisms of organic compounds secreted by plants, reduced nitrogen-rich fertilizer usage, increased solubilization of soil nutrients, increased size and/or quantity of carbon- and water-binding soil-mineral aggregates, improved retention and dispersion of water and/or nutrients in soil, reduced soil salinity and pollutant content, and increased microbial biomass and necromass in soil.

In one embodiment, the microbe-based soil treatment composition is applied to a lagoon or livestock manure pond, wherein the soil treatment composition can increase the rate of waste decomposition, improve the separation and dewatering of manure, and control methanogenic microorganisms present in the manure. Accordingly, the methods can contribute to reduced GHG emissions from manure and provide for efficient treatment, processing and recycling of manure as crop fertilizers.

In one embodiment, the microbe-based soil treatment composition is applied to soil or land experiencing drought and/or aquifer depletion, wherein the soil treatment composition increases the wettability of soil, improves retention and dispersion of water in soil, improves the drainage and dispersion of pooling water in hydrophobic soils, reduces water loss due to evaporation, and/or increases water penetration into deeper soil layers and groundwater sources, such as aquifers. Accordingly, the methods can contribute to improved water use efficiency, improved drought management, and recharging of depleted aquifers.

The methods can further comprise applying materials to enhance microbe, plant, and/or soil health during application (e.g., adding germination enhancers, prebiotics, nutrients and/or mineral sources). In one embodiment, these additional materials can include, for example, sources of magnesium, phosphate, nitrogen, potassium, selenium, calcium, sulfur, iron, copper, zinc, proteins, vitamins and/or carbon; prebiotics, such as, for example, one or more of biochar, kelp extract, fulvic acid, chitin, humate and humic acid; and/or rock dust.

The methods and compositions of the subject invention can be used either alone or in combination with additional components, such as herbicides, pesticides, soil amendments and/or fertilizers, such as, e.g., Mosaic's MicroEssentials SZ®, or Ostara's Synchro® or Crystal Green®. Preferably, the additional components are non-toxic and environmentally-friendly. The exact materials and the quantities thereof can be determined by, for example, a grower or soil scientist having the benefit of the subject disclosure.

### Modes of Application

As used herein, “applying” a composition or product to a site refers to contacting a composition or product with a site such that the composition or product can have an effect on that site. The effect can be due to, for example, microbial growth and colonization, and/or the action of a metabolite, enzyme, biosurfactant or other microbial growth by-product, and/or activity of an accelerator substance. The mode of application depends upon the formulation of the composition, and can include, for example, spraying, pouring, sprinkling, injecting, spreading, mixing, dunking, fogging and misting. Formulations can include, for example, liquids, dry and/or wettable powders, flowable powders, dusts, granules, pellets, emulsions, microcapsules, steaks, oils, gels, pastes and/or aerosols. In an exemplary embodiment, the subject soil treatment composition is applied after the composition has been prepared by, for example, dissolving the composition in water.

In one embodiment, the site to which the composition is applied is the soil (or rhizosphere) in which plants will be planted or are growing (e.g., a crop, a field, an orchard, a grove, a pasture/prairie or a forest). The compositions of the subject invention can be pre-mixed with irrigation fluids, wherein the compositions percolate through the soil and can be delivered to, for example, the roots of plants to influence the root microbiome.

In one embodiment, the compositions are applied to soil surfaces, with or without water, where the beneficial effect of the soil application can be activated by rainfall, sprinkler, flood, or drip irrigation.

In one embodiment, the composition is applied to a plant or plant part. The composition can be applied directly thereto as a seed treatment, or to the surface of a plant or plant part (e.g., to the surface of the roots, tubers, stems, flowers, leaves, fruit, or flowers). In a specific embodiment, the composition is contacted with one or more roots of the plant. The composition can be applied directly to the roots, e.g., by spraying or dunking the roots, and/or indirectly, e.g., by administering the composition to the soil in which the plant grows (or the rhizosphere). The composition can be applied to the seeds of the plant prior to or at the time of planting, or to any other part of the plant and/or its surrounding environment.

In one embodiment, wherein the method is used in a large scale setting, such as in a crop, a muck field, a citrus grove, a pasture or prairie, a forest, a sod or turf farm, or another agricultural crop, the method can comprise administering the composition into a tank connected to an irrigation system used for supplying water, fertilizers, pesticides or other liquid compositions. Thus, the plant and/or soil surrounding the plant can be treated with the composition via, for example, soil injection, soil drenching, using a center pivot irrigation system, with a spray over the seed furrow, with micro-jets, with drench sprayers, with boom sprayers, with sprinklers and/or with drip irrigators. Advantageously, the method is suitable for treating hundreds or more acres of land.

In one embodiment, wherein the method is used in a smaller scale setting, the method can comprise pouring the composition (mixed with water and other optional additives) into the tank of a handheld lawn and garden sprayer and spraying soil or another site with the composition. The composition can also be mixed into a standard handheld watering can and poured onto a site.

Soil, plants and/or their environments can be treated at any point during the process of cultivating the plant. For example, the composition can be applied to the soil prior to, concurrently with, or after the time when seeds or plants are planted therein. It can also be applied at any point thereafter during the development and growth of the plant, including when the plant is flowering, fruiting, and during and/or after abscission of leaves.

### Crop Management

The subject compositions and methods can be useful for enhancing plant health, growth and/or yields, including that of livestock feed crops; enhancing sequestration of carbon in soil, vegetation and microbial biomass; and/or reducing fertilizer usage.

In one embodiment, methods are provided for enhancing plant health, growth and/or yields wherein one or more microorganisms is contacted with the plant and/or its surrounding environment. The method can comprise applying a soil treatment composition of the subject invention.

In certain embodiments, the microorganisms of the composition work synergistically with one another to enhance health, growth and/or yields in plants.

In one embodiment, the method can enhance plant health, growth and/or yields by enhancing root health and growth. More specifically, in one embodiment, the methods can be used to improve the properties of the rhizosphere in which a plant's roots are growing, for example, the nutrient and/or moisture retention and dispersion properties.

Additionally, in one embodiment, the method can be used to inoculate a plant's rhizosphere with one or more beneficial microorganisms. For example, in preferred embodiments, the microbes of the soil treatment composition can colonize the rhizosphere and provide multiple benefits to the plants that result in enhanced utilization and storage of carbon via enhanced growth and/or health of both aerial and subterranean plant tissue.

In some embodiments, the subject methods increase the above- and below-ground biomass of plants, which includes, for example, increased foliage volume, increased stem and/or trunk diameter, enhanced root growth and/or density, and/or increased total numbers of plants

Advantageously, in certain embodiments, the subject methods can be used to enhance health, growth and/or yields in plants having compromised immune health due to an infection from a pathogenic agent or from an environmental stressor, such as, for example, drought. Thus, in certain embodiments, the subject methods can also be used for improving the immune health, or immune response, of plants.

For example, the plant may be affected by a pathogenic strain of Pseudomonas (e.g., P. savastanoi, P. syringae pathovars); Ralsionia solanacearum; Agrobacterium (e.g., A. tumefaciens); Xanthomonas (e.g., X. oryzae pv. Oryzae, X. campestris pathovars, X. axonopodis pathovars); Erwinia (e.g., E. amylovora); Xylella (e.g., X. fastidiosa); Dickeya (e.g., D. dadantii and D. solani); Pectobacterium (e.g., P. carotovorum and P. atrosepticum); Clavibacter (e.g., C. michiganensis and C. sepedonicus); Candidatus Liberibacter asiaticus; Pantoea; Burkholderia; Acidovorax; Streptomyces; Spiroplasma; and/or Phytoplasma; as well as huanglongbing (HLB, citrus greening disease), citrus canker disease, citrus bacterial spot disease, citrus variegated chlorosis, brown rot, citrus root rot, citrus and black spot disease.

In one embodiment, the methods are used to enhance the health, growth and/or yields of citrus trees affected by citrus greening disease and/or citrus canker disease.

In certain preferred embodiments, the compositions and methods are useful for enhancing plant immune health without directly targeting pests through pesticidal activity.

The present invention can be used to enhance health, growth and/or yields of plants and/or crops in, for example, agriculture, horticulture, greenhouses, landscaping, and the like. The present invention can also be used for improving one or more qualities of soil, thereby enhancing the performance of the soils for agricultural, home and gardening purposes. Furthermore, the present invention can be used in pasture management, as well as in professional turf, ornamental and landscape management.

In certain embodiments, the soil treatment composition may also be applied so as to promote colonization of the roots and/or rhizosphere as well as the vascular system of the plant in order to enhance plant health and vitality. Thus, growth of nutrient-fixing microbes such as Rhizobium and/or Mycorrhizae can be promoted, as well as other beneficial endogenous and exogenous microbes, and/or their by-products that promote crop growth, health and/or yield. The microbe-based product can also support a plant's vascular system by, for example, entering and colonizing said vascular system and contributing metabolites, and nutrients important to plant health and productivity.

In yet another embodiment, the method can be used to fight off, outcompete and/or discourage colonization of the rhizosphere by soil microorganisms that are deleterious or that might compete with beneficial soil microorganisms.

In one embodiment, the method can be used for enhancing penetration of beneficial molecules through the outer layers of root cells, for example, at the root-soil interface of the rhizosphere.

In certain embodiments, this is achieved via the presence of a biosurfactant, either produced in situ by a microorganism of the subject soil treatment composition, or applied directly to the soil. Biosurfactants according to the subject invention can help reduce the surface tension of water, allowing for increased uptake of nutrients and water into the plant roots, as well as increased efficiency when circulating water and nutrients throughout the plant's extremities. This is useful even for tall trees that must fight gravity, as well as plants that might be diseased and experience vascular constriction due to, e.g., pathogenic biofilms. Furthermore, this mode of action can enhance not only the transport of beneficial materials into a plant, but also the exports of toxins, free radicals and waste products from the plant with more efficiency.

In certain embodiments, the subject biosurfactants are particularly useful for these purposes due to their small micelle size. In certain embodiments, a sophorolipid biosurfactant has a micelle size less than 50 nm, less than 25 nm or less than 15 nm. Advantageously, this allows for enhanced penetration of small spaces and/or pores, such as those found between cells and in biofilm matrices.

In certain embodiments, the subject method enhances plant utilization and storage of carbon, which is achieved by enhancing the accumulation of degradation-resistant organic polymers in plant tissue and/or soil. In some embodiments, the accumulation of degradation-resistant organic polymers is further enhanced by utilizing plants that have been modified to produce greater-than-normal amounts of a particular degradation-resistant organic polymer.

In certain embodiments, the degradation-resistant organic polymers are polysaccharides, polyaromatics and/or polyesters. Examples found in plants include, but are not limited to, suberin, cutin, cutan, and lignins. In preferred embodiments, due to, for example, the complex nature of their chemical structure, the degradation-resistant organic polymers are not readily biodegradable for, e.g., at least 1 year, at least 5 years, at least 25 years, at least 100 years, or even at least 1,000 years after the death of the plant.

In addition to enhancing plant utilization and storage of carbon, colonization of the roots and/or soil by the microbes of the subject composition can also increase soil carbon sequestration. In certain embodiments, increasing soil carbon sequestration is achieved by enhancing the growth of plant roots in the soil and/or increasing accumulation of degradation-resistant organic polymers in the plant roots.

In one embodiment, the methods and compositions according to the subject invention lead to an increase in one or more of: root length, root density, root mass, stalk diameter, plant height, canopy density, chlorophyll content, flower count, bud count, bud size, bud density, leaf surface area, oil content, fruit count, fruit size, fiber content, and/or nutrient uptake of a plant, by at least 1%, 5%, 10%, 20%, 30%, 40%, 50%, 60%, 70%, 80%, 90%, 100%, 150%, 200%, or more, compared to a plant growing in an untreated environment.

In certain embodiments, the subject compositions and methods can be used for improving agricultural fertilization practices. For example, in preferred embodiments, the microbes of the soil treatment composition can colonize the rhizosphere and/or work synergistically with other free-living and endophytic nutrient-fixing microbes to provide enhanced solubilization of nutrients in the soil, such that the nutrients are more bioavailable for plant root uptake.

In some embodiments, the subject invention can be used to reduce and/or replace a chemical or synthetic fertilizer, wherein the composition comprises a microorganism capable of fixing, solubilizing, mobilizing and/or increasing the bioavailability and/or root uptake of nitrogen, potassium, K₂O, phosphorous, P₂O₅, and/or other micronutrients in soil, such as, e.g., S, Zn, B, and Mn. In other words, the subject invention can be useful for improving nutrient use efficiency and/or treating/preventing plant nutrient deficiencies.

In one embodiment, the method can be used to provide the plant with phosphorus in the form of phosphates. In certain embodiments, Wickerhamomyces anomalus can produce phytase, an enzyme that is capable of converting phytic acid present in soil into plant-bioavailable (e.g., root-absorbable) phosphates. Accordingly, the method can be used to treat and/or prevent a phosphorus deficiency in a plant, reduce phosphorus usage in fertilizers, and/or reduce phosphorus runoff into water sources.

In one embodiment, the method can be used for improving nitrogen use efficiency, as well as reducing nitrous oxide emissions. In some embodiments, microbes such as B. subtilis B4, B. amyloliquefaciens “B. amy”, Meyerozyma guilliermondii and Meyerozyma caribbica (e.g., strain MEC14XN) can fix nitrogen. In some embodiments, the subject soil treatment compositions can be used synergistically with free-living and/or endophytic nitrogen fixers, such as Klebsiella, Azotobacter and/or Azospirillum, for microbial facilitated nutrient release and cycling. Thus, in some embodiments, improved nitrogen use efficiency, reduced nitrous oxide emissions, and reduced nitrogen runoff into water sources can be achieved by replacing some or all nitrogen-rich fertilizers and/or increasing soil nitrogen uptake by plant roots using soil treatment compositions according to the subject invention.

### Soil Management

The subject compositions and methods can be useful for enhancing soil health through the rebuilding of degraded soils, prevention of soil degradation, improving soil aggregate formation, improving dispersion of water, nutrients and salts in soil, removing pollutants from soil, and even controlling soil-borne pests.

In certain embodiments, the soil treatment compositions are utilized in methods for preserving, rebuilding and/or regenerating soils in need thereof. In certain preferred embodiments, the soil comprises at least 10% organic matter by volume, at least 50% organic matter, or at least 80% organic matter. In a specific embodiment, the soil is classified as muck soil, mucky peat and/or peat soil.

In certain embodiments, the subject invention can be used to improve any number of qualities in any other type of soil, for example, clay, sandy, silty, peaty, chalky, loam soil, and/or combinations thereof. Furthermore, the methods and compositions can be used for improving the quality of dry, waterlogged, porous, depleted, compacted soils and/or combinations thereof.

The methods can be utilized in, for example, agricultural fields, pastures, orchards, prairies, plots, and/or forests. The methods can also be utilized in areas containing soil that is significantly uninhabitable by plant life, for example, soils that have been over-cultivated and/or where crop rotation has not been implemented or has been insufficient to retain the soil's fertility; soils that have eroded or subsided; soils that have been polluted by over-treatment with pesticides, fertilizers and/or herbicides; soils with high salinity; soils that have been polluted by dumping, or chemical or hydrocarbon spills; and/or soils in areas damaged by natural or anthropogenic causes, including fire, flooding, pest infestation, development (e.g. commercial, residential or urban building), digging, mining, logging, livestock rearing, and other causes.

Advantageously, the methods can help enhance agricultural yields, even in depleted or damaged soils; restore depleted greenspaces, such as pastures, forests, wetlands and prairies; and restore uncultivatable land so that it can be used for farming, reforestation and/or natural regrowth of plant ecosystems.

In certain embodiments, the methods comprise a step of characterizing the soil type and/or soil health status prior to treating the soil according to the subject methods. Accordingly, the method can also comprise tailoring the composition in order to meet a specific soil type and/or soil health need. Methods of characterizing soils are known in the agronomic arts.

In some embodiments, the microorganisms of the soil treatment composition colonize the rhizosphere and convert root exudates and digested organic matter into bulky, carbon-rich microbial biomass and necromass (dead cells).

In certain embodiments, the subject methods enhance SOC sequestration via, for example, increased above- and below-ground plant biomass, increased microbial biomass and/or necromass, and/or increased size and/or stability of soil aggregates. Furthermore, in certain embodiments, the methods can slow and/or stop soil profile degradation and/or erosion in areas where soil subsidence is occurring. Preferably, in some embodiments, the methods actually contribute to an increase in the depth of the soil profile.

Additionally, in certain embodiments, the subject methods can reduce the soil-borne emission of greenhouse gases, such as carbon dioxide, methane and nitrous oxide, which are caused by, for example, the decomposition of soil by low carbon use efficiency (CUE) microbes.

In certain embodiments, the methods of the subject invention further comprise applying one or more “accelerators” to the soil prior to, simultaneously with, and/or after the application of the soil treatment composition, such that the accelerator is available to the microorganisms of the soil treatment composition.

As used herein, an “accelerator” is any compound or substance that, when applied in the presence of the subject compositions, further decreases the rate of soil subsidence, increases the depth of the soil profile, increases the sequestration of SOC, enhances the health and/or growth of plant biomass, and/or decreases the rate of atmospheric carbon dioxide and other GHGs emitted from soil, compared to application of the soil treatment composition without the accelerator.

In one embodiment, the accelerator is a food source for the microorganisms. Non-limiting examples of food source accelerators include humic acid, kelp extract, fulvic acid, molasses, and mill mud.

In certain embodiments, the food source is one that is not typically found in the soil being treated, thereby providing a more diverse source of nutrients to the soil microorganisms. In some embodiments, the food source can be chosen based on the preferences of the particular microbe(s) in the soil treatment composition.

Advantageously, in some embodiments, the increased diversity of food resources encourages the diversification of the soil microbiome, which can lead to a reduction in the number of low CUE microorganisms microbes to decompose soil matter and produce GHGs. Additionally, in some embodiments, by increasing the availability of food resources for microbial consumption, the demand for carbon substrates is reduced, thereby reducing the production of enzymes that degrade labile carbon by soil microorganisms.

In one embodiment, the accelerator is a source of minerals and/or trace elements. In a specific embodiment, the minerals and/or trace elements are in the form of rock dust comprising finely crushed rock (also referred to as, e.g., rock minerals, rock flour, rock powder, stone dust, and/or mineral fines). Preferably, the rock dust is made up of basaltic rocks and/or silicate rocks that release elements such as calcium, magnesium, potassium, phosphorus and/or iron when weathering, or dissolving, in soil.

Advantageously, in some embodiments, the minerals and/or trace elements provide bioavailable micronutrients to enhance the health and/or growth of plants as well as microbes growing in the soil. In some embodiments, the minerals and/or trace elements facilitate the formation of carbon-mineral soil aggregates, the stability of which can be further enhanced by the microorganisms of the subject invention and/or plant root mass.

In some embodiments, the minerals and/or trace elements react with soil components to reduce carbon dioxide and/or nitrous oxide emissions from soil. For example, in one embodiment, the rock dust dissolves, reacting with carbon dioxide and capturing it in the form of carbon-storage molecules such as bicarbonates, calcium carbonate, and carbonate ions. In another embodiment, the rock dust alters the pH of the soil (i.e., increases it) as it weathers. Lower pH environments tend to inactivate diazotrophic N₂O reductase, which functions to reduce N₂O to N₂. Increasing the pH can allow for N₂O reductase to regain activity, thereby increasing the reduction of N₂O to N₂ and decreasing N₂O emissions.

In some embodiments, the methods are used in combination with existing soil preservation practices, such as no-till or low-till farming, crop rotation, and/or the planting of off-season cover crops.

Advantageously, the subject compositions and methods can help re-build soil resources that are traditionally considered non-renewable, while suppressing and/or averting soil GHG emissions and reducing the need for synthetic fertilizers.

In certain embodiments, the compositions and methods of the subject invention can be used for removing pollutants from soil, improving the nutrient content and availability of soil, improving drainage, dispersion and/or moisture retention properties of soil, improving nutrient retention and/or dispersion in soil, improving the salinity of soil, improving the diversity of the soil microbiome, and/or controlling a soil-borne pest. Other improvements can include adding bulk and/or structure to soils that have been eroded by wind and/or water, as well as preventing and/or delaying erosion of soil by wind and/or water.

Microbial biomass, whether active or inactive, provides organic matter that improves the physical structure of soils by, for example, adding bulk; helps reduce the erosion of soils by water and wind; and can increase the water retention capacity of soil, particularly porous, sandy soils. Furthermore, active and decaying microbial biomass improves the aeration, and thus water/nutrient infiltration, of heavy and compacted soils.

Other benefits of microbial biomass to soil include providing a nutrient source (e.g. nitrogen, phosphorus, potassium, sulfur, etc.) for plants as well as other soil microorganisms, dissolution of insoluble soil minerals to increase their bioavailability to plant roots due to, for example, favorable cation exchange capacity, regulation of soil temperature, and buffering of pesticide, herbicide, and other heavy metal residues.

In certain embodiments, the method results in removal and/or reduction of pollutants from soil, including remediation of soils contaminated with hydrocarbons. In some embodiments, the pollutants are degraded directly by the applied microorganisms of the composition. In some embodiments, the growth by-products of the microorganisms, e.g., biosurfactants, facilitate degradation of the pollutants, and can chelate and form a complex with ionic and nonionic metals to release them from the soil. Soil pollutants include, for example, residual fertilizers, pesticides, herbicides, fungicides, hydrocarbons, chemicals (e.g., dry cleaning treatments, urban and industrial wastes), benzene, toluene, ethylbenzene, xylene, and heavy metals.

In some embodiments, the microbial growth by-products, such as biosurfactants, serve as emulsifiers, increasing the oil-water interface of hydrocarbon pollutants by forming stable microemulsions with them. The result is an increase in the mobility and bioavailability of the pollutants for decomposing microorganisms.

The methods can further comprise supplying oxygen and/or nutrients to the microorganisms by circulating aqueous solutions through the soils, thus stimulating the applied microorganisms, as well as naturally-occurring soil microorganisms, to degrade the pollutants and/or produce pollutant-degrading growth by-products. In some embodiments, the polluted soil is combined with nonhazardous organic amendments such as manure or agricultural wastes. The presence of these organic materials supports the development of a rich microbial population and elevated temperature characteristics of composting. Thus, the rate of bioremediation can be increased.

In one embodiment, the composition comprises and/or is applied concurrently with biochar, or charcoal that is produced by, for example, pyrolysis of crop waste and other biomass in the absence of oxygen. In certain embodiments, biochar acts synergistically with the microorganisms of the soil treatment composition, providing an additional soil carbon source and facilitating the solubilization of nutrients, growth of larger roots, and growth of more robust plant biomass. In some embodiments, the biochar can adsorb and desorb fertilizer, thereby providing a slow-release application and reducing risk of leaching and denitrification before plant uptake.

In certain embodiments, the method results in improvement in the soil nutrient content and availability to plant roots. Biosurfactants enhance mobility of metals in soil to plants. Furthermore, microbial biomass, including live and inactive biomass, provide sources of nutrients, such as nitrogen, phosphorous and potassium (NPK), amino acids, vitamins, proteins and lipids.

In certain embodiments, the method results in improved salinity of soil by reducing the salt content. Saline soils contain sufficient neutral soluble salts to adversely affect the growth of most crop plants. Soluble salts most commonly present are the chlorides and sulfates of sodium, calcium and magnesium. Nitrates may be present rarely, while many saline soils contain appreciable quantities of gypsum (CaSO₄, 2H₂O).

When leached with low-salt water, some saline soils tend to disperse, resulting in low permeability to water and air, particularly when the soils are heavy clays. The presence of microorganisms and/or biosurfactants improves the mobility of salts and/or ions, thereby facilitating drainage of salts into depths below plant root zones.

In certain embodiments, the methods can also help improve soil microbiome diversity by promoting colonization of the soil and plant roots growing therein with beneficial soil microorganisms. Growth of nutrient-fixing microbes, such as rhizobium and/or mycorrhizae, can be promoted, as well as other endogenous and applied microbes, thereby increasing the number of different species within the soil microbiome.

In some embodiments, the methods are used for controlling above-ground and below-ground pests. In some embodiments, the method can be useful for controlling pests such as arthropods, nematodes, protozoa, bacteria, fungi, and/or viruses. In some embodiments, the method can be useful for modulating a plant's immune system to activate the plant's innate defenses against pests.

In one embodiment, the methods and compositions according to the subject invention lead to an increase in SOC in an area of soil, by at least 1%, 5%, 10%, 20%, 30%, 40%, 50%, 60%, 70%, 80%, 90%, 100%, 150%, 200%, or more, compared to similar untreated areas.

In one embodiment, the methods and compositions according to the subject invention lead to an increase in depth of soil profile by at least 1%, 5%, 10%, 20%, 30%, 40%, 50%, 60%, 70%, 80%, 90%, 100%, 150%, 200%, or more, compared to similar untreated areas.

In one embodiment, the methods and compositions according to the subject invention lead to a decrease in soil-borne emissions of GHG, such as CO2, N2O and/or CH4, by at least 1%, 5%, 10%, 20%, 30%, 40%, 50%, 60%, 70%, 80%, 900, 100%, or more, compared to similar untreated soil.

In one embodiment, the methods and compositions according to the subject invention lead to a decrease in soil pollutants by at least 1%, 5%, 10%, 20%, 30%, 40%, 50%, 60%, 70%, 80%, 90%, 100%, or more, compared to similar untreated soil.

In one embodiment, the methods and compositions according to the subject invention lead to a decrease in rhizosphere salinity by at least 1%, 5%, 10%, 20%, 30%, 40%, 50%, 60%, 70%, 80%, 90%, 100%, or more, compared to similar untreated soil.

### Water Management

In certain embodiments, the subject compositions and methods can be used for improving agricultural WUE, which can also reduce the total water consumption of livestock production by increasing the WUE of feed crop cultivation.

In certain embodiments, crop WUE can be improved via improved soil water retention due to increased microbial biomass and necromass in soil, which serves as a “sponge.” In certain embodiments, increased soil-mineral aggregates can also facilitate water retention via, for example, ionic interactions. Advantageously, in some embodiments, the methods help reduce agricultural water consumption, even in drought. Furthermore, reducing the water consumption required for growing feed crops, the total water consumption required for feeding livestock is also reduced.

In certain embodiments, microbial biosurfactants, whether produced in situ by microorganisms and/or applied individually, can decrease the tendency of water to pool, improve the adherence or wettability of soil, resulting in more thorough hydration of soil. This is particularly useful in the case of flood irrigation methods, which can lead to pooling water that stands on the surface and evaporates.

Improved wettability also promotes better root system health, as there are fewer zones of desiccation (or extreme dryness) inhibiting proper root growth and better availability of applied nutrients as chemical and micro-nutrients are more thoroughly made available and distributed. Furthermore, there can be fewer zones of extreme moisture, which can lead to root rot and the spread of certain pathogenic pests.

The more uniform distribution of water in soil made possible by enhanced wettability also prevents water from accumulating or getting trapped above optimal penetration levels, thereby mitigating anaerobic conditions that inhibit the free exchange of oxygen and carbon. When the composition is applied, a more porous or breathable soil is established.

In one embodiment, the methods and compositions according to the subject invention lead to a decrease in water consumption and/or an increase in WUE for crop production and/or livestock production by at least 1%, 5%, 10%, 20%, 30%, 40%, 50%, 60%, 70%, 80%, 90%, 100%, or more, compared to similar untreated crops.

Furthermore, in certain embodiments, the subject compositions and methods can be used for reversing and/or slowing aquifer depletion and groundwater scarcity (or, “recharging” the aquifers). Aquifer or groundwater depletion, as used herein, refers to a long-term water-level decline caused by, for example, sustained groundwater pumping.

Groundwater depletion is a serious problem in many areas of the world, including, for example, the plains and desert regions of the United States. Where surface water, such as lakes and rivers, are scarce or inaccessible, groundwater supplies municipal and agricultural water needs. Groundwater depletion can cause lowering of the water table, drying of wells, reduced water in streams and lakes, reduced water quality (e.g., saltwater intrusion), increased pumping costs, and even land subsidence. Particularly in warm areas and in times of drought, a charged aquifer is crucial for supplying drinking water and irrigation for crops.

In certain embodiments of the subject invention, methods for recharging an aquifer are provided, wherein a soil treatment composition according to the subject invention is applied to land in an area experiencing aquifer depletion. The land can then be watered manually, and/or water can simply be provided to the soil naturally via rainfall.

In certain embodiments, the treatment composition comprises a biosurfactant and/or a microorganism that produces a biosurfactant. In certain preferred embodiments, the treatment composition comprises a sophorolipid biosurfactant and/or a microorganism capable of producing a sophorolipid.

Similar to the benefits provided to crops, the amphiphilic properties of biosurfactants can enhance the dispersion of water throughout the soil in a way that allows water to more efficiently recharge groundwater stores. By improving the wettability of soil, the soil is more receptive to water so that the water is less likely to pool and/or evaporate in warmer weather. Instead, the water can penetrate the soil, including soils that are hydrophobic by nature, or have become hydrophobic due to, for example, long-term aquifer depletion.

In certain embodiments, the method further comprises monitoring the water levels within an aquifer treated according to the subject invention over time. For example, the method can comprise monitoring for changes in the water table level using standard methods in the hydrological arts.

In one embodiment, the methods and compositions according to the subject invention lead to an increase in groundwater levels and/or an increase in the water table by at least 1%, 5%, 10%, 20/s, 30%, 40%, 50%, 60%, 70%, 80%, 90%, 100%, or more, compared to similar untreated areas. In other embodiments, the rate of water table lowering is reduced by at least 1%, 5%, 10%, 20%, 30%, 40%, 50%, 60%, 70%, 80%, 90%, 100%.

### Livestock Management

In certain embodiments, the subject compositions and methods can be used for reducing the carbon footprint and/or carbon intensity of livestock production by reducing atmospheric greenhouse gas emissions resulting from livestock feed production, as well as livestock manure.

In some embodiments, the soil treatment composition is applied to land on which plants utilized for livestock feed are grown. In some embodiments, the farmland comprises a grass including, for example, bluegrasses, Bermuda grasses, fescues, buffalo grasses, and others. Other forage, such as forbs, weeds and shrubs, as well as feed crops such as corn, oats and barley, can also be treated with the soil treatment composition. In some embodiments, the composition can be applied to rangelands including, in some cases, land where prescribed burns have been performed to promote revegetation.

In one embodiment, the subject methods can be used for reducing the carbon footprint of producing grain-fed livestock, as well as grain-finished livestock, wherein the grains are reduced-carbon grains, and/or by-products thereof (e.g., distiller's dried grains with solubles, DDGS), that are produced according to the agricultural aspect of the subject methods.

In one embodiment, the method can increase the feed value of grasses compared with conventionally-grown, high carbon footprint grains that are produced for feeding and/or finishing livestock. By enhancing the marketability of pasture- or grass-fed livestock, and/or by feeding livestock grain that has been produced using reduced-carbon footprint methods, the subject invention can reduce the carbon footprint of the livestock feed industry by reducing land use change, reducing the manufacture and use of fertilizers and pesticides, and reducing the operation of fossil fuel burning agricultural machinery, including that used for processing and transport of feed.

This can be achieved by, for example, enhancing vegetative carbon utilization and storage in tracts of farmland, increasing carbon sequestration in soil, reducing soil-based GHG emissions, improving agricultural nitrogen-based fertilization practices, improving biodiversity in soil microbiota, and improving agricultural soil management.

In certain embodiments, enhanced vegetative carbon utilization can be in the form of, for example, increased foliage in plants, increased stem and/or trunk diameter, enhanced root growth, and/or increased numbers of plants per unit of area.

In certain embodiments, increased soil sequestration can be in the form of, for example, increased growth of plant roots (e.g., length and density), increased uptake by microorganisms of organic compounds secreted by plants (including secretions from plant roots), and improved microbial colonization of soil.

In certain embodiments, the methods can reduce the amount of GHG such as methane, carbon dioxide and/or nitrous oxide/precursors thereof emitted from soil.

In certain embodiments, the method further comprises making the plants produced according to the subject methods available to a livestock animal such that the livestock animal ingests the plants. In one embodiment, the livestock animal is placed on a tract of farmland treated according to the subject method for free-range grazing. In one embodiment, the plants are harvested from treated farmland and provided to the animal as reduced-carbon footprint fodder, grain and/or other forms of loose feed.

In one embodiment, a combination of feeding methods is utilized, for example, as is customary when producing grain-finished livestock.

In one embodiment, the method reduces the carbon footprint of the livestock industry by enhancing the health and/or productivity of livestock animals in ways that reduce GHG emissions resulting from digestion, manure and large-scale production of livestock.

These benefits can include, for example, improved feed efficiency, which results in improved animal health and fertility, improved quantity and nutritional quality of meat and dairy products, and reduced dependency on high-carbon footprint feed crops, such as conventionally-produced corn. One specific and important benefit of improved feed efficiency is increased feed nitrogen usage, which results in reduced ammonia and nitrous oxide produced in the digestive system and waste products of livestock. In addition, improved feed efficiency can result in improved animal productivity, meaning less feed and/or fewer GHG-emitting animals are required to produce a given amount of product.

In one embodiment, the animal husbandry aspect can further comprise applying the soil treatment composition directly to livestock manure to facilitate increased decomposition of manure by the microorganisms while reducing the amount of GHGs emitted therefrom. Manure contains two components that can lead to GHG emissions during storage and processing: organic matter that can be converted into methane emissions, and nitrogen that leads indirectly to nitrous oxide emissions. Methane is released when methanogenic bacteria decompose the organic material in the manure as it is being held in lagoons, tailing ponds or holding tanks. Additionally, nitrogen in the form of ammonia (NH₃) is released from manure and urine during storage and processing. Ammonia can later be transformed into nitrous oxide.

In some embodiments, applying the composition to manure further contributes to a reduced carbon footprint, as the manure can then be utilized in place of synthetic nitrogen-rich fertilizers for farmland that will eventually become feed for the livestock animals.

Advantageously, in certain embodiments, the subject invention provides solutions that improve the environmental sustainability of producing and consuming meat, dairy, and other animal-based products by, for example, promoting feed crop and pastureland growth and vitality; improving the nutritional content of farmland soils; promoting improved soil moisture and water use efficiency; enhancing soil microbiome diversity; reducing fertilizer usage; increasing the feed value of grasses while reducing grain dependency; reducing enteric GHG emissions from livestock animals and manure; improving feed to muscle conversion; improving productivity of livestock animals, e.g., quantity and nutritional quality of meat and milk; and others.

In certain specific embodiments, the composition can be administered directly to a manure lagoon, waste pond, tailing pond, tank or other storage facility where livestock manure is stored and/or treated. Advantageously, in some embodiments, the microorganisms in the composition can facilitate an increased decomposition rate for the manure while reducing the amount of methane and/or nitrous oxide emitted therefrom. Furthermore, in some embodiments, applying the composition to the manure enhances the value of the manure as an organic fertilizer due to the ability of the microorganisms to inoculate the soil to which the manure is applied. The microbes then grow and, for example, improve soil biodiversity, enhance rhizosphere properties, and enhance plant growth and health.

Reducing the Carbon Footprint and/or Carbon Intensity

A “carbon footprint” may be defined herein as a measure of the total amount of carbon dioxide (CO₂) and other GHGs emitted directly or indirectly by a human activity or accumulated over the full life cycle of a product or service. As just one example, a product that requires transportation over many miles by truck (e.g., harvested feed grains) may have a larger carbon footprint than an alternative product that does not require transportation (e.g., grass growing in a pasture).

Carbon footprints can be calculated using a Life Cycle Assessment (LCA) method, or can be restricted to the immediately attributable emissions from energy use of fossil fuels. A life cycle assessment (LCA, also known as life cycle analysis, ecobalance, and cradle-to-grave analysis) is the investigation and valuation of the environmental impacts of a given product or service caused or necessitated by its existence. The life cycle concept of the carbon footprint means that it is all-encompassing and includes all possible causes that give rise to carbon emissions. In other words, all direct (on-site, internal) and indirect emissions (off-site, external, embodied, upstream, downstream) need to be taken into account.

Normally, a carbon footprint is expressed as a CO₂ equivalent. Carbon dioxide equivalency is a quantity that describes, for a given mixture and amount of GHG, the amount of CO₂ that would have the same global warming potential (GWP), when measured over a specified timescale (generally, 100 years). Carbon dioxide equivalency thus reflects time-integrated radiative forcing. The carbon dioxide equivalency for a gas is obtained by multiplying the mass and the GWP of the gas. The following units are commonly used:
a) By the UN climate change panel IPCC: billion metric tonnes of CO₂ equivalent (GtCO₂ eq);
b) In industry: million metric tonnes of carbon dioxide equivalents (MMTCDE);
c) For vehicles: g of carbon dioxide equivalents/km (gCDE/km).

For example, the GWP for methane is 21 and for nitrous oxide 310. This means that emissions of 1 million metric tonnes of methane and nitrous oxide respectively is equivalent to emissions of 21 and 310 million metric tonnes of carbon dioxide.

Various methods exist in the art for calculating or estimating carbon footprints and may be employed in the subject invention.

Advantageously, in preferred embodiments, the subject invention can be useful for reducing the carbon footprint of producing livestock, which includes reducing the carbon footprint of producing forage-based, fodder-based and/or grain-based feed for livestock.

A “reduced carbon footprint” means a negative alteration in the amount of carbon dioxide and other GHGs emitted per unit time over the full life cycle of producing livestock feed and feeding livestock with said feed, through and until a livestock/animal-based product is ultimately consumed by human consumers. The negative alteration in CO₂ and/or other GHG emissions can be, for example, at least 0.25%, 0.5%, 1%, 5%, 10%, 15%, 20%, 25%, 30%, 35%, 40%, 45%, 50%, 55%, 60%, 65%, 70%, 75%, 80%, 85%, 90° h, 95% or 100%.

In some embodiments, the term “carbon footprint” is interchangeable herein with the terms “carbon intensity” and “emission intensity.” Emission intensity is the measure of the emission rate of a given GHG relative to the “intensity” of a specific activity or industrial process (e.g., burning of fuel, production of livestock animals, production of corn). The emissions intensity can include amount of emissions relative to, for example, amount of fuel combusted, yield of corn harvested, number of livestock animals produced, amount of an commercial product produced, total distance traveled, and/or number of economic units generated.

Emissions intensity is measured across the entire life cycle of a product. For example, the emissions intensity of fuels is calculated by compiling all of the GHG emissions emitted along the supply chain for a fuel, including all the emissions emitted in exploration, mining, collecting, producing, transporting, distributing, dispensing and burning the fuel.

In addition to reducing the carbon footprint and/or carbon intensity of agriculture and livestock production, in some embodiments, the subject invention can be used for reducing the number of carbon credits used by an operator involved in, e.g., agriculture, livestock production, forestry/reforestation, and wetland management.

Advantageously, the systems of the subject invention can increase the efficiency and reduce the financial and environmental costs of agriculture and livestock practices. In particular, the compositions and methods utilized according to the subject invention can help in preserving valuable natural resources, such as soil and water, while improving production of valuable plant and animal-based commodities.

### Monitoring of Factors Such as Soil Moisture, Microbial Population, and GHG

In some embodiments, the systems of the subject invention further involve the monitoring of various inputs and outputs of crop and livestock production. For example, following application of a soil treatment composition to a tract of land, factors such as water usage, soil moisture content and dispersion, fertilizer usage, soil nutrient content and dispersion, soil microbial populations, soil organic content, generation of GHG emissions, fossil fuel usage, plant growth, health and yields, and even the presence of pests, can be monitored. Accordingly, the system can be adjusted throughout implementation to account for changes in these factors and make appropriate adjustments to the inputs in real time.

Monitoring can be performed in place and/or via wireless methods using known methods in the art. For example, in some embodiments, fixed location sensors can be used to detect GHG emissions from a field and transmit data to a network for remote monitoring. Nanocrystal detection assays, such as those described in U.S. Patent Application Publication No. US-2020-0102602-A1, incorporated herein by reference, can be used for monitoring microbial populations and other analytes in soil. Soil moisture can be monitored by either manually sampling soil at various depths, or by devices such as tensiometers, gypsum blocks, granular matrix sensors, and time domain reflectometers (TDR).

In some embodiments, monitoring comprises performing one or more measurements to assess the effect of the methods of the subject invention on the generation and/or reduction in generation of GHGs, and/or the accumulation of carbon in soil. In one embodiment, the method comprises simply measuring the depth of the soil profile to determine whether the soil profile has decreased, increased, and/or remained stable after treatment with the subject compositions over time.

In certain embodiments, the measurements assess the effect of the methods of the subject invention on the generation and/or reduction in generation of GHGs and/or on the accumulation of SOC in plants and/or soil.

Measurements and/or monitoring can be conducted at a certain time point after application of the soil treatment composition to the site. In some embodiments, the measurements are conducted after about 1 week or less, 2 weeks or less, 3 weeks or less, 4 weeks or less, 30 days or less, 60 days or less, 90 days or less, 120 days or less, 180 days or less, and/or 1 year or less.

Furthermore, the measurements and/or monitoring can be repeated over time. In some embodiments, the measurements are repeated daily, weekly, monthly, bi-monthly, semi-monthly, semi-annually, and/or annually.

In certain embodiments, assessing GHG generation can take the form of measuring GHG emissions from a site. Gas chromatography with electron capture detection is commonly used for testing samples in a lab setting. In certain embodiments, GHG emissions can also be conducted in the field, using, for example, flux measurements and/or in situ soil probing. Flux measurements analyze the emission of gases from the soil surface to the atmosphere, for example, using chambers that enclose an area of soil and then estimate flux by observing the accumulation of gases inside the chamber over a period of time. Probes can be used to generate a soil gas profile, starting with a measurement of the concentration of the gases of interest at a certain depth in the soil, and comparing it directly between probes and ambient surface conditions (Brummell and Siciliano 2011, at 118).

Measuring GHG emissions can also comprise other forms of direct emissions measurement, gas chromatography-mass spectrometry (GC-MS) and/or analysis of fuel input. Direct emissions measurements can comprise, for example, identifying polluting operational activities (e.g., fuel-burning automobiles) and measuring the emissions of those activities directly through Continuous Emissions Monitoring Systems (CEMS). Fuel input analysis can comprise calculating the quantity of energy resources used (e.g., amount of electricity, fuel, wood, biomass, etc., consumed) determining the content of, for example, carbon, in the fuel source, and applying that carbon content to the quantity of the fuel consumed to determine the amount of emissions.

In certain embodiments, carbon content of a site where plants are growing, e.g., agricultural site, crop, sod or turf farm, pasture/prairie or forest, can be measured by, for example, quantifying the aboveground and/or below-ground biomass of plants. In general, the carbon concentration of, for example, a tree, is assumed to be from about 40 to 50% of the biomass.

Biomass quantification can take the form of, for example, harvesting plants in a sample area and measuring the weight of the different parts of the plant before and after drying. Biomass quantification can also be carried out using non-destructive, observational methods, such as measuring, e.g., trunk diameter, height, volume, and other physical parameters of the plant. Remote quantification of biomass and/or GHG missions can also be used, such as, for example, laser profiling, optical sensors and/or drone analysis.

In some embodiments, carbon content of a site can further comprise sampling and measuring carbon content of litter, woody debris and/or soil of a sampling area. Soil, in particular, can be analyzed, for example, using dry combustion to determine percent total organic carbon (TOC); by potassium permanganate oxidation analysis for detecting active carbon; and by bulk density measurements (weight per unit volume) for converting from percent carbon to tons/acre.

Advantageously, the subject invention facilitates holistic approaches to solving pain points involved with agriculture and livestock production. Thus, in some embodiments, the invention provides an agricultural or agronomic program that is prescribed for a particular crop or tract of land, and that can be adjusted over time to meet changing conditions and/or changing goals for a grower or livestock producer.

In some embodiments, the aspects of the system can be centralized such that they are managed, facilitated and/or performed by a single entity. The entity can be a company or a person who manages, facilitates and/or performs all aspects of producing the microbial compositions, formulation and/or customization of the compositions, transportation and application of the compositions, and monitoring of inputs and outputs throughout the course of the treatment. The central entity can also utilize input and output data collected from a single customer and/or multiple customers to predict and formulate future adjustments to the prescribed agronomic or agricultural program.

In some embodiments, the central entity can serve as a general contractor, with sub-contractors performing one or more of the production, formulation/customization, transportation and application of the soil treatment compositions, as well as monitoring.

### Target Plants

As used here, the term “plant” includes, but is not limited to, any species of woody, ornamental or decorative, crop or cereal, fruit plant or vegetable plant, flower or tree, macroalga or microalga, phytoplankton and photosynthetic algae (e.g., green algae Chlamydomonas reinhardtii). “Plant” also includes a unicellular plant (e.g., microalga) and a plurality of plant cells that are largely differentiated into a colony (e.g., volvox) or a structure that is present at any stage of a plant's development. Such structures include, but are not limited to, a fruit, a seed, a shoot, a stem, a leaf, a root, a flower petal, etc. Plants can be standing alone, for example, in a garden, or can be one of many plants, for example, as part of an orchard, crop or pasture.

As used herein, “crop plants” refer to any species of plant or alga, grown for profit and/or for sustenance for humans, animals or aquatic organisms, or used by humans (e.g., textile, cosmetics, and/or drug production), or viewed by humans for pleasure (e.g., flowers or shrubs in landscaping or gardens) or any plant or alga, or a part thereof, used in industry, commerce or education. Crop plants can be plants that can be obtained by traditional breeding and optimization methods or by biotechnological and recombinant methods, or combinations of these methods, including the transgenic plants and the plant varieties.

Types of crop plants that can benefit from application of the products and methods of the subject invention include, but are not limited to: row crops (e.g., corn, soy, sorghum, peanuts, potatoes, etc.), field crops (e.g., alfalfa, wheat, grains, etc.), tree crops (e.g., walnuts, almonds, pecans, hazelnuts, pistachios, etc.), citrus crops (e.g., orange, lemon, grapefruit, etc.), fruit crops (e.g., apples, pears, strawberries, blueberries, blackberries, etc.), turf crops (e.g., sod), ornamentals crops (e.g., flowers, vines, etc.), vegetables (e.g., tomatoes, carrots, etc.), vine crops (e.g., grapes, etc.), forestry (e.g., pine, spruce, eucalyptus, poplar, etc.), managed pastures (any mix of plants used to support grazing animals).

Additional examples of plants for which the subject invention is useful include, but are not limited to, cereals and grasses (e.g., wheat, barley, rye, oats, rice, maize, sorghum, corn), beets (e.g., sugar or fodder beets); fruit (e.g., grapes, strawberries, raspberries, blackberries, pomaceous fruit, stone fruit, soft fruit, apples, pears, plums, peaches, almonds, cherries or berries); leguminous crops (e.g., beans, lentils, peas or soya); oil crops (e.g., oilseed rape, mustard, poppies, olives, sunflowers, coconut, castor, cocoa or ground nuts); cucurbits (e.g., pumpkins, cucumbers, squash or melons); fiber plants (e.g., cotton, flax, hemp or jute); citrus fruit (e.g., oranges, lemons, grapefruit or tangerines); vegetables (e.g., spinach, lettuce, asparagus, cabbages, carrots, onions, tomatoes, potatoes or bell peppers); Lauraceae (e.g., avocado, Cinnamonium or camphor); and also tobacco, nuts, herbs, spices, medicinal plants, coffee, eggplants, sugarcane, tea, pepper, grapevines, hops, the plantain family, latex plants, cut flowers and ornamentals.

In certain embodiments, the crop plant is a citrus plant. Examples of citrus plants according to the subject invention include, but are not limited to, orange trees, lemon trees, lime trees and grapefruit trees. Other examples include Citrus maxima (Pomelo), Citrus medica (Citron), Citrus micrantha (Papeda), Citrus reticulata (Mandarin orange), Citrus paradisi (grapefruit), Citrus japonica (kumquat), Citrus australasica (Australian Finger Lime), Citrus australis (Australian Round lime), Citrus glauca (Australian Desert Lime), Citrus garrawayae (Mount White Lime), Citrus gracilis (Kakadu Lime or Humpty Doo Lime), Citrus inodora (Russel River Lime), Citrus warburgiana (New Guinea Wild Lime), Citrus wintersii (Brown River Finger Lime), Citrus halimii (limau kadangsa, limau kedut kera), Citrus indica (Indian wild orange), Citrus macroptera, and Citrus latipes, Citrus×aurantiifolia (Key lime), Citrus×aurantium (Bitter orange), Citrus×latfolia (Persian lime), Citrus×limon (Lemon), Citrus×limonia (Rangpur), Citrus×sinensis (Sweet orange), Citrus×tangerina (Tangerine), Imperial lemon, tangelo, orangelo, tangor, kinnow, kiyomi, Minneola tangelo, oroblanco, ugli, Buddha's hand, citron, bergamot orange, blood orange, calamondin, clementine, Meyer lemon, and yuzu.

In some embodiments, the crop plant is a relative of a citrus plant, such as orange jasmine, limeberry, and trifoliate orange (Citrus trifolata).

Additional examples of target plants include all plants that belong to the superfamily Viridiplantae, in particular monocotyledonous and dicotyledonous plants including fodder or forage legumes, ornamental plants, food crops, trees or shrubs selected from Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Arlocarpus spp., Asparagus officinalis, Avena spp. (e.g., A. sativa, A. fatua, A. byzantina, A. fatua var. sativa, A. hybrida), Averrhoa carambola, Bambusa sp., Benincasa hispida, Bertholletia excelsea, Beta vulgaris, Brassica spp. (e.g., B. napus, B. rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Cofea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g., E. guineensis, E. oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g., G. max, Soja hispida or Soja max), Gossypium hirsutum, Helianthus spp. (e.g., H. annuus), Hemerocallis fulva, Hibiscus spp., Hordeum spp. (e.g., H. vulgare), Ipomoea batatas, Juglans spp., Lactuca saliva, Lathyrus spp., Lens culinaris, Linum usitatissimum, Litchi chinensis, Lotus spp., Lufa acutangula, Lupinus spp., Luzula sylvatica, Lycopersicon spp. (e.g., L. esculentum, L. lycopersicum, L. pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g., O. sativa, O. latifolia), Panicum miliaceum, Panicum virgatum, Passilora edulis, Pastinaca saliva, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp. (e.g., Q. suber L), Raphanus sativus, Rheum rhabarbarum, Ribes spp., Ricinus communis, Rubus spp., Saccharum spp., Salir sp., Sambucus spp., Secale cereale, Sesamum spp., Sinapis sp., Solanum spp. (e.g., S. tuberosum, S. integrifolium or S. lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g., T. aestivum, T. durum, T. turgidum, T. hybernum, T. macha, T satvun, T. monococcum or T. vulgare), Tropaeolum minus, Tropaeolum majus, Vaccinium spp., Vicia spp., Vigna spp., Viola odorata, Vitis spp., Zea mays, Zizania palustris, Ziziphus spp., amongst others.

Target plants can also include, but are not limited to, corn (Zea mays), Brassica sp. (e.g., B. napus, B. rapa, B. juncea), particularly those Brassica species useful as sources of seed oil, alfalfa (Medicago saliva), rice (Oryza saliva), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Cofea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Target vegetable plants include tomatoes (Lycopersicon esculentum), lettuce (e.g., Lactuca saliva), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Conifers that may be employed in practicing the embodiments include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). Plants of the embodiments include crop plants (for example, corn, alfalfa, sunflower, Brassica, soybean, cotton, safflower, peanut, sorghum, wheat, millet, tobacco, etc.), such as corn and soybean plants.

Target turfgrasses include, but are not limited to: annual bluegrass (Poa annua); annual ryegrass (Lolium multiflorum); Canada bluegrass (Poa compressa); Chewings fescue (Festuca rubra); colonial bentgrass (Agrostis tenuis); creeping bentgrass (Agrostis palustris); crested wheatgrass (Agropyron desertorum); fairway wheatgrass (Agropyron cristatum); hard fescue (Festuca longifolia); Kentucky bluegrass (Poa pratensis); orchardgrass (Dactylis glomerate); perennial ryegrass (Lolium perenne); red fescue (Festuca rubra); redtop (Agrostis alba); rough bluegrass (Poa trivialis); sheep fescue (Festuca ovine); smooth bromegrass (Bromus inermis); tall fescue (Festuca arundinacea); timothy (Phleum pretense); velvet bentgrass (Agrostis canine); weeping alkaligrass (Puccinellia distans); western wheatgrass (Agropyron smithii); Bermuda grass (Cynodon spp.); St. Augustine grass (Stenotaphrum secundatum); zoysia grass (Zoysia spp.); Bahia grass (Paspalum notatum); carpet grass (Axonopus affinis); centipede grass (Eremochloa ophiuroides); kikuyu grass (Pennisetum clandesinum); seashore paspalum (Paspalum vaginatum); blue gramma (Bouteloua gracilis); buffalo grass (Buchloe dactyloids); sideoats gramma (Bouteloua curtipendula).

Further plants of interest include grain plants that provide seeds of interest, oil-seed plants, and leguminous plants. Seeds of interest include grain seeds, such as corn, wheat, barley, rice, sorghum, rye, millet, etc. Oil-seed plants include cotton, soybean, safflower, sunflower, Brassica, maize, alfalfa, palm, coconut, flax, castor, olive etc. Leguminous plants include beans and peas. Beans include guar, locust bean, fenugreek, soybean, garden beans, cowpea, mungbean, lima bean, fava bean, lentils, chickpea, etc. Further plants of interest include Cannabis (e.g., sativa, indica, and ruderalis) and industrial hemp.

All plants and plant parts can be treated in accordance with the invention. In this context, plants are understood as meaning all plants and plant populations such as desired and undesired wild plants or crop plants (including naturally occurring crop plants). Crop plants can be plants that can be obtained by traditional breeding and optimization methods or by biotechnological and recombinant methods, or combinations of these methods, including the transgenic plants and the plant varieties.

Plant tissue and/or plant parts are understood as meaning all aerial and subterranean parts and organs of the plants such as shoots, leaves, flowers, roots, needles, stalks, stems, fruits, seeds, tubers and rhizomes. The plant parts also include crop material and vegetative and generative propagation material, for example cuttings, tubers, rhizomes, slips and seeds.

### Soil Treatment Compositions

In certain embodiments, the subject invention provides soil treatment compositions comprising one or more soil-colonizing microorganisms and/or growth by-products thereof, such as biosurfactants, enzymes, polysaccharides and/or other metabolites. The composition may also comprise the fermentation broth/medium in which the microorganism(s) were produced.

In some embodiments, the microorganisms of the subject invention have a greater CUE than microbes already present in the soil to which they are applied. In some embodiments, the microorganisms of the subject composition are “high CUE,” meaning the percentage of carbon they allocate to biomass production is greater than the percentage allocated to respiration.

In certain embodiments, the microorganisms are bacteria, yeasts and/or fungi. In some embodiments, the composition comprises more than one type and/or species of microorganism. Advantageously, in some embodiments, the microorganisms colonize the rhizosphere and convert root exudates and digested organic matter into bulky, carbon-rich microbial biomass and necromass (dead cells).

In preferred embodiments, the microbe-based compositions according to the subject invention are non-toxic and can be applied in high concentrations without causing irritation to, for example, the skin or digestive tract of a human or other non-pest animal. Thus, the subject invention is particularly useful where application of the microbe-based compositions occurs in the presence of living organisms, such as growers and livestock.

In one embodiment, multiple microorganisms can be used together, where the microorganisms create a synergistic beneficial effect on plant and/or soil health.

The species and ratio of microorganisms and other ingredients in the composition can be customized and optimized for specific local conditions at the time of application, such as, for example, which soil type, plant and/or crop is being treated; what season, climate and/or time of year it is when a composition is being applied; and what mode and/or rate of application is being utilized. Thus, the composition can be customizable for any given site.

The microorganisms useful according to the subject invention can be, for example, non-plant-pathogenic strains of bacteria, yeast and/or fungi. These microorganisms may be natural, or genetically modified microorganisms. For example, the microorganisms may be transformed with specific genes to exhibit specific characteristics. The microorganisms may also be mutants of a desired strain. As used herein, “mutant” means a strain, genetic variant or subtype of a reference microorganism, wherein the mutant has one or more genetic variations (e.g., a point mutation, missense mutation, nonsense mutation, deletion, duplication, frameshift mutation or repeat expansion) as compared to the reference microorganism. Procedures for making mutants are well known in the microbiological art. For example, UV mutagenesis and nitrosoguanidine are used extensively toward this end.

In one embodiment, the microorganism is a yeast or fungus. Yeast and fungus species suitable for use according to the current invention, include Aureobasidium (e.g., A. pullulans) Blakeslea, Candida (e.g., C. apicola, C. bombicola, C. nodaensis), Cryptococcus, Debaryomyces (e.g., D. hansenit), Entomophthora, Hanseniaspora, (e.g., H. uvarum), Hansenula, Issatchenkia, Kuyveromyces (e.g., K. phaffii), Lentinula edodes, Mortierella, mycorrhizal fungi, Meyerozyma (M. guilliermondii, M. caribbica, M. aphidis), Penicillium, Phycomyces, Pichia (e.g., P. anomala, P. guilliermondii, P. occidentalis, P. kudriavzevii), Pleurotus spp. (e.g., P. ostreatus), Pseudozyma (e.g., P. aphidis), Saccharomyces (e.g., S. boulardii sequela, S. cerevisiae, S. torula), Starmerella (e.g., S. bombicola), Torulopsis, Trichoderma (e.g., T. guizhouse, T. reesei, T. harzianum, T. koningii, T. hamatum, T. viride), Ustilago (e.g., U. maydis), Wickerhamomyces (e.g., W. anomalus), Williopsis (e.g., W. mrakii), Zygosaccharomyces (e.g., Z. bailii), and others.

As used herein, “mycorrhizal fungi” includes any species of fungus that forms a non-parasitic mycorrhizal relationship with a plant's roots. The fungi can be ectomycorrhizal fungi and/or endomycorrhizal fungi, including subtypes thereof (e.g., arbuscular, ericoid, and orchid mycorrhizae).

Non-limiting examples of mycorrhizal fungi according to the subject invention include species belong to Glomeromycota, Basidiomycota, Ascomycota, Zygomycota, Helotiales, and Hymenochaetales, as well as Acaulospora spp. (e.g., A. alpina, A. brasiliensis, A. foveata), Amanita spp. (e.g., A. muscaria, A. phalloides), Amphinema spp. (e.g., A. byssoides, A. diadema, A. rugosum), Astraeus spp. (e.g., A. hygrometricum), Byssocorticium spp. (e.g., B. atrovirens), Byssoporia terrestris (e.g., B. terrestris sartoryi, B. terrestris lilacinorosea, B. terrestris aurantiaca, B. terrestris sublutea, B. terrestris parksii), Cairneyella spp. (e.g., C. variabilis), Cantherellus spp. (e.g., C. cibarius, C. minor, C. cinnabarinus, C. friesis), Cenococcum spp. (e.g., C. geophilum), Ceratobasidium spp. (e.g., C. cornigerum), Cortinarius spp. (e.g., C. austrovenetus, C. caperatus, C. violaceus), Endogone spp. (e.g., E. pisiformis), Entrophospora spp. (e.g., E. colombiana), Funneliformis spp. (e.g., F. mosseae), Gamarada spp. (e.g., G. debralockiae), Gigaspora spp. (e.g., G. gigantean, G. margarita), Glomus spp. (e.g., G. aggregatum, G. brasilianum, G. clarum, G. deserticola, G. etunicatum, G. fasciculatum G. intraradices, G. lamellosum, G. macrocarpum, G. monosporum, G. mosseae, G. versiforme), Gomphidius spp. (e.g., G. glutinosus), Hebeloma spp. (e.g., H. cylindrosporum), Hydnum spp. (e.g., H. repandum), Hymenoscyphus spp. (e.g., H. ericae), Inocybe spp. (e.g., I. bongardii, I. sindonia), Lactarius spp. (e.g., L. hygrophoroides), Lindtneria spp. (e.g., L. brevispora), Melanogaster spp. (e.g., M. ambiguous), Meliniomyces spp. (e.g., M. variabilis), Morchella spp., Mortierella spp. (e.g., M. polycephala), Oidiodendron spp. (e.g., O. maius), Paraglomus spp. (e.g., P. brasilianum), Paxillus spp. (e.g., P. involutus), Penicillium spp. (e.g., P. pinophilum, P. thomili), Peziza spp. (e.g., P. whitei), Pezoloma spp. (e.g., P. ericae); Phlebopus spp. (e.g., P. marginatus), Pioderma spp. (e.g., P. croceum), Pisolithus spp. (e.g., P. tinctorius), Pseudotomentella spp. (e.g., P. tristis), Rhizoctonia spp., Rhizodermea spp. (e.g., R. veluwensis), Rhizophagus spp. (e.g., R. irregularis), Rhizopogon spp. (e.g., R. luteorubescens, R. pseudoroseolus), Rhizoscyphus spp. (e.g., R. ericae), Russula spp. (e.g., R. livescens), Sclerocystis spp. (e.g., S. sinuosum), Scleroderma spp. (e.g., S. cepa, S. verrucosum), Scutellospora spp. (e.g., S. pellucida S. heterogama), Sebacina spp. (e.g., S. sparassoidea), Setcheliogaster spp. (e.g., S. tenuipes), Suillus spp. (e.g., S. luteus), Thanatephorus spp. (e.g., T. cucumeris), Thelephora spp. (e.g., T. terrestris), Tomentella spp. (e.g., T. badia, T. cinereoumbrina, T. erinalis, T. galzinii), Tomentellopsis spp. (e.g., T. echinospora), Trechispora spp. (e.g., T. hymenocystis, T. stellulata T. thelephora), Trichophaea spp. (e.g., T. abundans, T. woolhopeia), Tulasnella spp. (e.g., T. calospora), and Tylospora spp. (e.g., T. fibrillose).

In certain preferred embodiments, the subject invention utilizes endomycorrhizal fungi, including fungi from the phylum Glomeromycota and the genera Glomus, Gigaspora, Acaulospora, Sclerocystis, and Entrophospora. Examples of endomycorrhizal fungi include, but not are not limited to, Glomus aggregatum, Glomus brasilianum, Glomus clarum, Glomus deserticola, Glomus etunicatum, Glomus fasciculatum, Glomus intraradices (Rhizophagus irregularis), Glomus lamellosum, Glomus macrocarpum, Gigaspora margarita, Glomus monosporum, Glomus mosseae (Funneliformis mosseae), Glomus versiforme, Scutellospora heterogama, and Sclerocystis spp.

In certain embodiments, the microorganisms are bacteria, including Gram-positive and Gram-negative bacteria. The bacteria may be, for example Agrobacterium (e.g., A. radiobacter), Azotobacter (A. vinelandii, A. chroococcum), Azospirillum (e.g., A. brasiliensis), Bacillus (e.g., B. amyloliquefaciens, B. circulans, B. firmus, B. laterosporus, B. licheniformis, B. megaterium, B. mucilaginosus, B. polymyxa, B. subtilis (including strains B1, B2, B3 and B4), Brevibacillus laterosporus), Frateuria (e.g., F. aurantia), Microbacterium (e.g., M. laevaniformans), myxobacteria (e.g., Myxococcus xanthus, Stignatella aurantiaca, Sorangium cellulosum, Minicystis rosea), Paenibacillus polymyxa, Pantoea (e.g., P. agglomerans), Pseudomonas (e.g., P. aeruginosa, P. chlororaphis subsp. aureofaciens (Kluyver), P. putida), Rhizobium spp., Rhodospirillum (e.g., R. rubrum), Sphingomonas (e.g., S. paucimobilis), and/or Thiobacillus thiooxidans (Acidothiobacillus thiooxidans).

In certain embodiments, the microorganism is one that is capable of fixing and/or solubilizing nitrogen, potassium, phosphorous and/or other micronutrients in soil.

In one embodiment, the microbe helps provide the plant with phosphorus in the form of phosphates. W. anomalus can produce phytase, an enzyme that is capable of converting phytic acid present in soil into plant-bioavailable (e.g., root-absorbable) phosphates.

In one embodiment, the microorganism is a nitrogen-fixing microorganism, or a diazotroph, selected from species of, for example, B. amy, B. subtilis B4, Meyerozyma guilliermondii, Meyerozyma caribbica (e.g., strain MEC14XN), Azospirillum, Azotobacter, Chlorobiaceae, Cyanothece, Frankia, Klebsiella, rhizobia, Trichodesmium, and some Archaea. In a specific embodiment, the nitrogen-fixing bacterium is Azotobacter vinelandii.

In one embodiment, the microorganism is a potassium-mobilizing microorganism, or KMB, selected from, for example, Bacillus mucilaginosus, Frateuria aurantia, Wickerhamomyces anomalus or Glomus mosseae.

In one embodiment, the microorganism is a non-denitrifying microorganism capable of converting nitrous oxide from the atmosphere into nitrogen in the soil, such as, for example, Dyadobacter fermenters.

In one embodiment, a combination of microorganisms is used in the subject microbe-based composition, wherein the microorganisms work synergistically with one another to enhance plant biomass, and/or to enhance the properties of the rhizosphere.

In specific exemplary embodiments, the microbes utilized according to the subject invention are selected from one or more of: Trichoderma spp. (e.g., T. harzianum, T. viride, T. koningii, and T. guizhouse); Bacillus spp. (e.g., B. amyloliquefaciens, B. subtilis, B. megaterium, B. polymyxa, B. licheniformis, and Brevibacillus laterosporus); Meyerozyma guilliermondii; Meyerozyma caribbica; Pichia occidentalis; Pichia kudriavzevii; Wickerhamomyces anomalus; and Debaryomyces hansenii.

In one specific exemplary embodiment, the composition comprises a Bacillus bacterium, such as, e.g., B. amyloliquefaciens or B. subtilis, and a Trichoderma sp., such as, for example, T. harzianum (e.g., T. harzianum T-22).

In one embodiment, the composition comprises B. amyloliquefaciens NRRL B-67928 “B. amy.” A culture of the B. amyloliquefaciens “B. amy” microbe has been deposited with the Agricultural Research Service Northern Regional Research Laboratory (NRRL) Culture Collection, 1815 N. University St., Peoria, IL, USA. The deposit has been assigned accession number NRRL B-67928 by the depository and was deposited on Feb. 26, 2020.

In one embodiment, the composition comprises B. subtilis NRRL B-68031 “B4.” A culture of the B4 microbe has been deposited with the Agricultural Research Service Northern Regional Research Laboratory (NRRL) Culture Collection, 1815 N. University St., Peoria, IL, USA. The deposit has been assigned accession number NRRL B-68031 by the depository and was deposited on May 6, 2021.

In one embodiment, the composition comprises W. anomalus NRRL Y-68030. A culture of this microbe has been deposited with the Agricultural Research Service Northern Regional Research Laboratory (NRRL) Culture Collection, 1815 N. University St., Peoria, IL, USA. The deposit has been assigned accession number NRRL Y-68030 by the depository and was deposited on May 6, 2021.

In one embodiment, the composition comprises Meyerozyma caribbica strain “MEC14XN” NRRL ______. A culture of this microbe has been deposited with the Agricultural Research Service Northern Regional Research Laboratory (NRRL) Culture Collection, 1815 N. University St., Peoria, IL, USA. The deposit has been assigned accession number NRRL ______ by the depository and was deposited on ______.

The subject culture has been deposited under conditions that assure that access to the culture will be available during the pendency of this patent application to one determined by the Commissioner of Patents and Trademarks to be entitled thereto under 37 CFR 1.14 and 35 U.S.C 122. The deposit is available as required by foreign patent laws in countries wherein counterparts of the subject application, or its progeny, are filed. However, it should be understood that the availability of a deposit does not constitute a license to practice the subject invention in derogation of patent rights granted by governmental action.

Further, the subject culture deposit will be stored and made available to the public in accord with the provisions of the Budapest Treaty for the Deposit of Microorganisms, i.e., it will be stored with all the care necessary to keep it viable and uncontaminated for a period of at least five years after the most recent request for the furnishing of a sample of the deposit, and in any case, for a period of at least 30 (thirty) years after the date of deposit or for the enforceable life of any patent which may issue disclosing the culture. The depositor acknowledges the duty to replace the deposit should the depository be unable to furnish a sample when requested, due to the condition of the deposit. All restrictions on the availability to the public of the subject culture deposit will be irrevocably removed upon the granting of a patent disclosing it.

In a specific embodiment, the concentration of each microorganism included in the composition is 1×10⁶ to 1×10¹³ CFU/g, 1×10⁷ to 1×10¹² CFU/g, 1×10⁸ to 1×10¹¹ CFU/g, or 1×10⁹ to 1×10¹⁰ CFU/g of the composition.

In one embodiment, the total microbial cell concentration of the composition is at least 1×10⁶ CFU/g, including up to 1×10⁹ CFU/g, 1×10¹⁰, 1×10¹¹, 1×10¹² and/or 1×10¹³ or more CFU/g.

In one embodiment, the microorganisms of the subject composition comprise about 5 to 20% h of the total composition by weight, or about 8 to 15%, or about 10 to 12%.

The composition can comprise the leftover fermentation substrate and/or purified or unpurified growth by-products, such as enzymes, biosurfactants and/or other metabolites. The microbes can be live or inactive.

The microbes and microbe-based compositions of the subject invention have a number of beneficial properties that are useful for improving crop and livestock production. For example, the compositions can comprise products resulting from the growth of the microorganisms, such as biosurfactants, proteins and/or enzymes, either in purified or crude form.

The microbes can also be useful for promoting increased above- and below-ground plant biomass per plant, increased high-carbon content polymers in plant tissue, increased numbers of plants per unit of area, increased uptake by microorganisms of organic compounds secreted by plants, reduced nitrogen-rich fertilizer usage, increased size and/or quantity of carbon- and water-binding soil-mineral aggregates, improved retention and dispersion of water in soil, reduced soil salinity and pollutant content, and increased microbial biomass and necromass in soil. Furthermore, in some embodiments, the microorganisms can induce auxin production, enable solubilization, absorption and/or dispersion of nutrients in the soil, and protect plants from pests and pathogens.

In one embodiment, the microorganisms of the subject composition are capable of producing a biosurfactant. In another embodiment, biosurfactants can be produced separately by other microorganisms and applied, either in purified form or in crude form. Crude form biosurfactants can comprise, for example, biosurfactants and other products of cellular growth in the leftover fermentation medium resulting from cultivation of a biosurfactant-producing microbe. This crude form biosurfactant composition can comprise from about 0.001% to about 90%, about 25% to about 75%, about 30% to about 70%, about 35% to about 65%, about 40% to about 60%, about 45% to about 55%, or about 50% pure biosurfactant.

Biosurfactants form an important class of secondary metabolites produced by a variety of microorganisms such as bacteria, fungi, and yeasts. As amphiphilic molecules, microbial biosurfactants reduce the surface and interfacial tensions between the molecules of liquids, solids, and gases. Furthermore, the biosurfactants according to the subject invention are biodegradable, have low toxicity, are effective in solubilizing and degrading insoluble compounds in soil and can be produced using low cost and renewable resources. They can inhibit adhesion of undesirable microorganisms to a variety of surfaces, prevent the formation of biofilms, and can have powerful emulsifying and demulsifying properties. Furthermore, the biosurfactants can also be used to improve wettability and to achieve even solubilization and/or distribution of fertilizers, nutrients, and water in the soil.

Advantageously, biosurfactants can be useful for replacing and/or reducing the usage of synthetic, chemical and/or petroleum-based surfactants in agricultural products. Surfactants are often used as emulsifiers, dispersing agents, spreaders, stickers, solubilizers wetting agents, defoamers, drift reducers, deposition agents, and adjuvants for herbicides, pesticides and fertilizers. These surfactants can be toxic when used at high doses, and often persist in soil for extended periods of time without degrading. Specific non-limiting examples include linear alkylbenzenesulfonates, sodium serate, DDAC, CHAPS, sodium dodecyl sulfate, quaternary ammonium cationic compounds, CAPB, and monolaurin (glycerol monolaurate).

Biosurfactants according to the subject methods can be selected from, for example, low molecular weight glycolipids (e.g., sophorolipids, cellobiose lipids, rhamnolipids, mannosylerythritol lipids and trehalose lipids), lipopeptides (e.g., surfactin, iturin, fengycin, arthrofactin and lichenysin), flavolipids, phospholipids (e.g., cardiolipins), fatty acid esters, and high molecular weight polymers such as lipoproteins, lipopolysaccharide-protein complexes, and polysaccharide-protein-fatty acid complexes.

The composition can comprise one or more biosurfactants at a concentration of 0.001% to 10%, 0.01% to 5%, 0.05% to 2%, and/or from 0.1% to 1% by weight.

The composition can comprise the fermentation medium containing a live and/or an inactive culture, the purified or crude form growth by-products, such as biosurfactants, enzymes, and/or other metabolites, and/or any residual nutrients.

The product of fermentation may be used directly, with or without extraction or purification. If desired, extraction and purification can be easily achieved using standard extraction and/or purification methods or techniques described in the literature.

The microorganisms in the composition may be in an active or inactive form, or in the form of vegetative cells, reproductive spores, mycelia, hyphae, conidia or any other form of microbial propagule. The composition may also contain a combination of any of these microbial forms.

In one embodiment, when a combination of strains of microorganism are included in the composition, the different strains of microbe are grown separately and then mixed together to produce the composition.

Advantageously, in accordance with the subject invention, the composition may comprise the medium in which the microbes were grown. The composition may be, for example, at least, by weight, 1%, 5%, 10%, 25%, 50%, 75%, or 100% growth medium. The amount of biomass in the composition, by weight, may be, for example, anywhere from 0% to 100% inclusive of all percentages therebetween.

In one embodiment, the composition is preferably formulated for application to soil, seeds, whole plants, or plant parts (including, but not limited to, roots, tubers, stems, flowers and leaves). In certain embodiments, the composition is formulated as, for example, liquid, dust, granules, microgranules, pellets, wettable powder, flowable powder, emulsions, microcapsules, oils, or aerosols.

To improve or stabilize the effects of the composition, it can be blended with suitable adjuvants and then used as such or after dilution, if necessary. In preferred embodiments, the composition is formulated as a liquid, a concentrated liquid, or as dry powder or granules that can be mixed with water and other components to form a liquid product. In one embodiment, the composition can comprise glucose (e.g., in the form of molasses), in addition to an osmoticum substance, to ensure optimum osmotic pressure during storage and transport of the dry product.

Further components can be added to the composition, for example, buffering agents, carriers, other microbe-based compositions produced at the same or different facility, viscosity modifiers, preservatives, nutrients for microbe growth, tracking agents, biocides, other microbes, surfactants, emulsifying agents, lubricants, solubility controlling agents, pH adjusting agents, preservatives, stabilizers and ultra-violet light resistant agents.

The pH of the composition should be suitable for the microorganism of interest as well as for the soil environment to which it will be applied. In some embodiments, the pH is about 2.0 to about 10.0, about 2.0 to about 9.5, about 2.0 to about 9.0, about 2.0 to about 8.5, about 2.0 to about 8.0, about 2.0 to about 7.5, about 2.0 to about 7.0, about 3.0 to about 7.5, about 4.0 to about 7.5, about 5.0 to about 7.5, about 5.5 to about 7.0, about 6.5 to about 7.5, about 3.0 to about 5.5, about 3.25 to about 4.0, or about 3.5. Buffers, and pH regulators, such as carbonates and phosphates, may be used to stabilize pH near a preferred value.

Optionally, the composition can be stored prior to use. The storage time is preferably short. Thus, the storage time may be less than 60 days, 45 days, 30 days, 20 days, 15 days, 10 days, 7 days, 5 days, 3 days, 2 days, 1 day, or 12 hours. In a preferred embodiment, if live cells are present in the product, the product is stored at a cool temperature such as, for example, less than 20° C., 15° C., 10° C., or 5° C.

The microbe-based compositions may be used without further stabilization, preservation, and storage, however. Advantageously, direct usage of these microbe-based compositions preserves a high viability of the microorganisms, reduces the possibility of contamination from foreign agents and undesirable microorganisms, and maintains the activity of the by-products of microbial growth.

In other embodiments, the composition (microbes, growth medium, or microbes and medium) can be placed in containers of appropriate size, taking into consideration, for example, the intended use, the contemplated method of application, the size of the fermentation vessel, and any mode of transportation from microbe growth facility to the location of use. Thus, the containers into which the microbe-based composition is placed may be, for example, from 1 pint to 1,000 gallons or more. In certain embodiments the containers are 1 gallon, 2 gallons, 5 gallons, 25 gallons, or larger.

The compositions can be used in combination with other agricultural compounds and/or crop management systems. In one embodiment, the composition can optionally comprise, or be applied with, for example, natural and/or chemical pesticides, repellants, herbicides, fertilizers, water treatments, non-ionic surfactants and/or soil amendments. Preferably, however, the composition does not comprise and/or is not used with benomyl, dodecyl dimethyl ammonium chloride, hydrogen dioxide/peroxyacetic acid, imazilil, propiconazole, tebuconazole, or triflumizole.

If the composition is mixed with compatible chemical additives, the chemicals are preferably diluted with water prior to addition of the subject composition.

In one embodiment, the subject compositions are compatible for use with agricultural compounds characterized as antiscalants, such as, e.g., hydroxyethylidene diphosphonic acid;
bactericides, such as, e.g., streptomycin sulfate and/or Galltrol® (A. radiobacter strain K84);
biocides, such as, e.g., chlorine dioxide, didecyldimethyl ammonium chloride, halogenated heterocyclic, and/or hydrogen dioxide/peroxyacetic acid;
fertilizers, such as, e.g., N-P-K fertilizers, calcium ammonium nitrate 17-0-0, potassium thiosulfate, nitrogen (e.g., 10-34-0, Kugler KQ-XRN, Kugler KS-178C, Kugler KS-2075, Kugler LS 6-24-6S, UN 28, UN 32), and/or potassium;
fungicides, such as, e.g., chlorothalonil, manicozeb hexamethylenetetramine, aluminum tris, azoxystrobin, Bacillus spp. (e.g., B. licheniformis strain 3086, B. subtilis, B. subtilis strain QST 713), benomyl, boscalid, pyraclostrobin, captan, carboxin, chloroneb, chlorothalonil, copper culfate, cyazofamid, dicloran, dimethomorph, etridiazole, thiophanate-methyl, fenamidone, fenarimol, fludioxonil, fluopicolide, flutolanil, iprodione, mancozeb, maneb, mefanoxam, fludioxonil, mefenoxam, metalaxyl, myclobutanil, oxathiapiprolin, pentachloronitrolbenzene (quintozene), phosphorus acid, propamocarb, propanil, pyraclostrobin, Reynoutria sachalinensis, Streptomyces spp. (e.g., S. griseoviridis strain K61, S. lydicus WYEC 108), sulfur, urea, thiabendazole, thiophanate methyl, thiram, triadimefon, triadimenol, and/or vinclozolin;
growth regulators, such as, e.g., ancymidol, chlormequat chloride, diaminozide, paclobutrazol, and/or uniconazole;
herbicides, such as, e.g., glyphosate, oxyfluorfen, and/or pendimethalin;
insecticides, such as, e.g., acephate, azadirachtin, B. thuringiensis (e.g., subsp. israelensis strain AM 65-52), Beauveria bassiana (e.g., strain GHA), carbaryl, chlorpyrifos, cyantraniliprole, cyromazine, dicofol, diazinon, dinotefuran, imidacloprid, Isaria fumosorosea (e.g., Apopka strain 97), lindane, and/or malathion;
water treatments, such as, e.g., hydrogen peroxide (30-35%), phosphonic acid (5-20%), and/or sodium chlorite;
as well as glycolipids, lipopeptides, deet, diatomaceous earth, citronella, essential oils, mineral oils, garlic extract, chili extract, and/or any known commercial and/or homemade pesticide that is determined to be compatible by the skilled artisan having the benefit of the subject disclosure.

Preferably, the composition does not comprise and/or is not applied simultaneously with, or within 7 to 10 days before or after, application of the following compounds: benomyl, dodecyl dimethyl ammonium chloride, hydrogen dioxide/peroxyacetic acid, imazilil, propiconazole, tebuconazole, or triflumizole.

In certain embodiments, the compositions and methods can be used to enhance the effectiveness of other compounds, for example, by enhancing the penetration of a pesticidal compound into a plant or pest, or enhancing the bioavailability of a nutrient to plant roots. The microbe-based products can also be used to supplement other treatments, for example, antibiotic treatments. Advantageously, the subject invention helps reduce the amount of antibiotics that must be administered to a crop or plant in order to be effective at treating and/or preventing bacterial infection.

### Growth of Microbes According to the Subject Invention

The subject invention utilizes methods for cultivation of microorganisms and production of microbial metabolites and/or other by-products of microbial growth. The subject invention further utilizes cultivation processes that are suitable for cultivation of microorganisms and production of microbial metabolites on a desired scale. These cultivation processes include, but are not limited to, submerged cultivation/fermentation, solid state fermentation (SSF), and modifications, hybrids and/or combinations thereof.

As used herein “fermentation” refers to cultivation or growth of cells under controlled conditions. The growth could be aerobic or anaerobic. In preferred embodiments, the microorganisms are grown using SSF and/or modified versions thereof.

In one embodiment, the subject invention provides materials and methods for the production of biomass (e.g., viable cellular material), extracellular metabolites (e.g. small molecules and proteins), residual nutrients and/or intracellular components (e.g. enzymes and other proteins).

The microbe growth vessel used according to the subject invention can be any fermenter or cultivation reactor for industrial use. In one embodiment, the vessel may have functional controls/sensors or may be connected to functional controls/sensors to measure important factors in the cultivation process, such as pH, oxygen, pressure, temperature, humidity, microbial density and/or metabolite concentration.

In a further embodiment, the vessel may also be able to monitor the growth of microorganisms inside the vessel (e.g., measurement of cell number and growth phases). Alternatively, a daily sample may be taken from the vessel and subjected to enumeration by techniques known in the art, such as dilution plating technique. Dilution plating is a simple technique used to estimate the number of organisms in a sample. The technique can also provide an index by which different environments or treatments can be compared.

In one embodiment, the method includes supplementing the cultivation with a nitrogen source. The nitrogen source can be, for example, potassium nitrate, ammonium nitrate ammonium sulfate, ammonium phosphate, ammonia, urea, and/or ammonium chloride. These nitrogen sources may be used independently or in a combination of two or more.

The method can provide oxygenation to the growing culture. One embodiment utilizes slow motion of air to remove low-oxygen containing air and introduce oxygenated air. In the case of submerged fermentation, the oxygenated air may be ambient air supplemented daily through mechanisms including impellers for mechanical agitation of liquid, and air spargers for supplying bubbles of gas to liquid for dissolution of oxygen into the liquid.

The method can further comprise supplementing the cultivation with a carbon source. The carbon source can be a carbohydrate, such as glucose, sucrose, lactose, fructose, trehalose, mannose, mannitol, and/or maltose; organic acids such as acetic acid, fumaric acid, citric acid, propionic acid, malic acid, malonic acid, and/or pyruvic acid; alcohols such as ethanol, propanol, butanol, pentanol, hexanol, isobutanol, and/or glycerol; fats and oils such as soybean oil, canola oil, rice bran oil, olive oil, corn oil, sunflower oil, sesame oil, and/or linseed oil; etc. These carbon sources may be used independently or in a combination of two or more.

In one embodiment, growth factors and trace nutrients for microorganisms are included in the medium. This is particularly preferred when growing microbes that are incapable of producing all of the vitamins they require. Inorganic nutrients, including trace elements such as iron, zinc, copper, manganese, molybdenum and/or cobalt may also be included in the medium. Furthermore, sources of vitamins, essential amino acids, and microelements can be included, for example, in the form of flours or meals, such as corn flour, or in the form of extracts, such as yeast extract, potato extract, beef extract, soybean extract, banana peel extract, and the like, or in purified forms. Amino acids such as, for example, those useful for biosynthesis of proteins, can also be included.

In one embodiment, inorganic salts may also be included. Usable inorganic salts can be potassium dihydrogen phosphate, dipotassium hydrogen phosphate, disodium hydrogen phosphate, magnesium sulfate, magnesium chloride, iron sulfate, iron chloride, manganese sulfate, manganese chloride, zinc sulfate, lead chloride, copper sulfate, calcium chloride, sodium chloride, calcium carbonate, and/or sodium carbonate. These inorganic salts may be used independently or in a combination of two or more.

In some embodiments, the method for cultivation may further comprise adding additional acids and/or antimicrobials in the medium before, and/or during the cultivation process. Antimicrobial agents or antibiotics are used for protecting the culture against contamination.

Additionally, antifoaming agents may also be added to prevent the formation and/or accumulation of foam during submerged cultivation.

The pH of the mixture should be suitable for the microorganism of interest. Buffers, and pH regulators, such as carbonates and phosphates, may be used to stabilize pH near a preferred value.

When metal ions are present in high concentrations, use of a chelating agent in the medium may be necessary.

The microbes can be grown in planktonic form or as biofilm. In the case of biofilm, the vessel may have within it a substrate upon which the microbes can be grown in a biofilm state. The system may also have, for example, the capacity to apply stimuli (such as shear stress) that encourages and/or improves the biofilm growth characteristics.

The pH of the culture should be suitable for the microorganism of interest as well as for the soil environment to which the composition will be applied. In some embodiments, the pH is about 2.0 to about 10.0, about 2.0 to about 9.5, about 2.0 to about 9.0, about 2.0 to about 8.5, about 2.0 to about 8.0, about 2.0 to about 7.5, about 2.0 to about 7.0, about 3.0 to about 7.5, about 4.0 to about 7.5, about 5.0 to about 7.5, about 5.5 to about 7.0, about 6.5 to about 7.5, about 3.0 to about 5.5, about 3.25 to about 4.0, or about 3.5. Buffers, and pH regulators, such as carbonates and phosphates, may be used to stabilize pH near a preferred value.

In one embodiment, the method of cultivation is carried out at about 5° to about 100° C., about 15° to about 60° C., about 20° to about 50° C., about 20° to about 45° C., about 25° to about 40° C., about 25° to about 37° C., about 25° to about 35° C., about 30° to about 35° C., about 24° to about 28° C., or about 22° to about 25° C. In one embodiment, the cultivation may be carried out continuously at a constant temperature. In another embodiment, the cultivation may be subject to changing temperatures.

In one embodiment, the equipment used in the method and cultivation process is sterile. The cultivation equipment such as the reactor/vessel may be separated from, but connected to, a sterilizing unit, e.g., an autoclave. The cultivation equipment may also have a sterilizing unit that sterilizes in situ before starting the inoculation. Air can be sterilized by methods know in the art. For example, the ambient air can pass through at least one filter before being introduced into the vessel. In other embodiments, the medium may be pasteurized or, optionally, no heat at all added, where the use of low water activity and low pH may be exploited to control undesirable bacterial growth.

In one embodiment, the subject invention further provides a method for producing microbial metabolites such as, for example, biosurfactants, enzymes, proteins, ethanol, lactic acid, beta-glucan, peptides, metabolic intermediates, polyunsaturated fatty acid, and lipids, by cultivating a microbe strain of the subject invention under conditions appropriate for growth and metabolite production; and, optionally, purifying the metabolite. The metabolite content produced by the method can be, for example, at least 20%, 30%, 40%, 50%, 60%, 70%, 80%, or 90%.

The microbial growth by-product produced by microorganisms of interest may be retained in the microorganisms or secreted into the growth medium. The medium may contain compounds that stabilize the activity of microbial growth by-product.

The biomass content of the fermentation medium may be, for example, from 5 g/l to 180 g/l or more, or from 10 g/l to 150 g/l.

The cell concentration may be, for example, at least 1×10⁶ to 1×10¹³, 1×10⁷ to 1×10¹², 1×10⁸ to 1×10¹¹, or 1×10⁹ to 1×10¹⁰ CFU/ml.

The method and equipment for cultivation of microorganisms and production of the microbial by-products can be performed in a batch, a quasi-continuous process, or a continuous process.

In one embodiment, all of the microbial cultivation composition is removed upon the completion of the cultivation (e.g., upon, for example, achieving a desired cell density, or density of a specified metabolite). In this batch procedure, an entirely new batch is initiated upon harvesting of the first batch.

In another embodiment, only a portion of the fermentation product is removed at any one time. In this embodiment, biomass with viable cells, spores, conidia, hyphae and/or mycelia remains in the vessel as an inoculant for a new cultivation batch. The composition that is removed can be a cell-free medium or contain cells, spores, or other reproductive propagules, and/or a combination of thereof. In this manner, a quasi-continuous system is created.

Advantageously, the method does not require complicated equipment or high energy consumption. The microorganisms of interest can be cultivated at small or large scale on site and utilized, even being still-mixed with their media.

Advantageously, the microbe-based products can be produced in remote locations. The microbe growth facilities may operate off the grid by utilizing, for example, solar, wind and/or hydroelectric power.

### Preparation of Microbe-Based Products

One microbe-based product of the subject invention is simply the fermentation medium containing the microorganisms and/or the microbial metabolites produced by the microorganisms and/or any residual nutrients. The product of fermentation may be used directly without extraction or purification. If desired, extraction and purification can be easily achieved using standard extraction and/or purification methods or techniques described in the literature.

The microorganisms in the microbe-based products may be in an active or inactive form, or in the form of vegetative cells, reproductive spores, conidia, mycelia, hyphae, or any other form of microbial propagule. The microbe-based products may also contain a combination of any of these forms of a microorganism.

In one embodiment, different strains of microbe are grown separately and then mixed together to produce the microbe-based product. The microbes can, optionally, be blended with the medium in which they are grown and dried prior to mixing.

In one embodiment, the different strains are not mixed together, but are applied to a plant and/or its environment as separate microbe-based products.

The microbe-based products may be used without further stabilization, preservation, and storage. Advantageously, direct usage of these microbe-based products preserves a high viability of the microorganisms, reduces the possibility of contamination from foreign agents and undesirable microorganisms, and maintains the activity of the by-products of microbial growth.

Upon harvesting the microbe-based composition from the growth vessels, further components can be added as the harvested product is placed into containers or otherwise transported for use. The additives can be, for example, buffers, carriers, other microbe-based compositions produced at the same or different facility, viscosity modifiers, preservatives, nutrients for microbe growth, surfactants, emulsifying agents, lubricants, solubility controlling agents, tracking agents, solvents, biocides, antibiotics, pH adjusting agents, chelators, stabilizers, ultra-violet light resistant agents, other microbes and other suitable additives that are customarily used for such preparations.

In one embodiment, buffering agents including organic and amino acids or their salts, can be added. Suitable buffers include citrate, gluconate, tartarate, malate, acetate, lactate, oxalate, aspartate, malonate, glucoheptonate, pyruvate, galactarate, glucarate, tartronate, glutamate, glycine, lysine, glutamine, methionine, cysteine, arginine and a mixture thereof. Phosphoric and phosphorous acids or their salts may also be used. Synthetic buffers are suitable to be used but it is preferable to use natural buffers such as organic and amino acids or their salts listed above.

In a further embodiment, pH adjusting agents include potassium hydroxide, ammonium hydroxide, potassium carbonate or bicarbonate, hydrochloric acid, nitric acid, sulfuric acid or a mixture.

In one embodiment, additional components such as an aqueous preparation of a salt, such as sodium bicarbonate or carbonate, sodium sulfate, sodium phosphate, sodium biphosphate, can be included in the formulation.

In certain embodiments, an adherent substance can be added to the composition to prolong the adherence of the product to plant parts. Polymers, such as charged polymers, or polysaccharide-based substances can be used, for example, xanthan gum, guar gum, levan, xylinan, gellan gum, curdlan, pullulan, dextran and others.

In preferred embodiments, commercial grade xanthan gum is used as the adherent. The concentration of the gum should be selected based on the content of the gum in the commercial product. If the xanthan gum is highly pure, then 0.001% (w/v—xanthan gum/solution) is sufficient.

In one embodiment, glucose, glycerol and/or glycerin can be added to the microbe-based product to serve as, for example, an osmoticum during storage and transport. In one embodiment, molasses can be included.

In one embodiment, prebiotics can be added to and/or applied concurrently with the microbe-based product to enhance microbial growth. Suitable prebiotics, include, for example, kelp extract, fulvic acid, chitin, biochar, humate and/or humic acid. In a specific embodiment, the amount of prebiotics applied is about 0.1 L/acre to about 0.5 L/acre, or about 0.2 L/acre to about 0.4 L/acre.

In one embodiment, specific nutrients are added to and/or applied concurrently with the microbe-based product to enhance microbial inoculation and growth. These can include, for example, soluble potash (K₂O), magnesium, sulfur, boron, iron, manganese, and/or zinc. The nutrients can be derived from, for example, potassium hydroxide, magnesium sulfate, boric acid, ferrous sulfate, manganese sulfate, and/or zinc sulfate.

Optionally, the product can be stored prior to use. The storage time is preferably short. Thus, the storage time may be less than 60 days, 45 days, 30 days, 20 days, 15 days, 10 days, 7 days, 5 days, 3 days, 2 days, 1 day, or 12 hours. In a preferred embodiment, if live cells are present in the product, the product is stored at a cool temperature such as, for example, less than 20° C., 15° C., 10° C., or 5° C.

### Local Production of Microbe-Based Products

In certain embodiments of the subject invention, a microbe growth facility produces fresh, high-density microorganisms and/or microbial growth by-products of interest on a desired scale. The microbe growth facility may be located at or near the site of application. The facility produces high-density microbe-based compositions in batch, quasi-continuous, or continuous cultivation.

The microbe growth facilities of the subject invention can be located at the location where the microbe-based product will be used (e.g., a citrus grove). For example, the microbe growth facility may be less than 300, 250, 200, 150, 100, 75, 50, 25, 15, 10, 5, 3, or 1 mile from the location of use.

Because the microbe-based product can be generated locally, without resort to the microorganism stabilization, preservation, storage and transportation processes of conventional microbial production, a much higher density of microorganisms can be generated, thereby requiring a smaller volume of the microbe-based product for use in the on-site application or which allows much higher density microbial applications where necessary to achieve the desired efficacy. This allows for a scaled-down bioreactor (e.g., smaller fermentation vessel, smaller supplies of starter material, nutrients and pH control agents), which makes the system efficient and can eliminate the need to stabilize cells or separate them from their culture medium. Local generation of the microbe-based product also facilitates the inclusion of the growth medium in the product. The medium can contain agents produced during the fermentation that are particularly well-suited for local use.

Locally-produced high density, robust cultures of microbes are more effective in the field than those that have remained in the supply chain for some time. The microbe-based products of the subject invention are particularly advantageous compared to traditional products wherein cells have been separated from metabolites and nutrients present in the fermentation growth media. Reduced transportation times allow for the production and delivery of fresh batches of microbes and/or their metabolites at the time and volume as required by local demand.

The microbe growth facilities of the subject invention produce fresh, microbe-based compositions, comprising the microbes themselves, microbial metabolites, and/or other components of the medium in which the microbes are grown. If desired, the compositions can have a high density of vegetative cells or propagules, or a mixture of vegetative cells and propagules.

In one embodiment, the microbe growth facility is located on, or near, a site where the microbe-based products will be used (e.g., a citrus grove), for example, within 300 miles, 200 miles, or even within 100 miles. Advantageously, this allows for the compositions to be tailored for use at a specified location. The formula and potency of microbe-based compositions can be customized for specific local conditions at the time of application, such as, for example, which soil type, plant and/or crop is being treated; what season, climate and/or time of year it is when a composition is being applied; and what mode and/or rate of application is being utilized.

Advantageously, distributed microbe growth facilities provide a solution to the current problem of relying on far-flung industrial-sized producers whose product quality suffers due to upstream processing delays, supply chain bottlenecks, improper storage, and other contingencies that inhibit the timely delivery and application of, for example, a viable, high cell-count product and the associated medium and metabolites in which the cells are originally grown.

Furthermore, by producing a composition locally, the formulation and potency can be adjusted in real time to a specific location and the conditions present at the time of application. This provides advantages over compositions that are pre-made in a central location and have, for example, set ratios and formulations that may not be optimal for a given location.

The microbe growth facilities provide manufacturing versatility by their ability to tailor the microbe-based products to improve synergies with destination geographies. Advantageously, in preferred embodiments, the systems of the subject invention harness the power of naturally-occurring local microorganisms and their metabolic by-products to improve GHG management.

The cultivation time for the individual vessels may be, for example, from 1 to 7 days or longer. The cultivation product can be harvested in any of a number of different ways.

Local production and delivery within, for example, 24 hours of fermentation results in pure, high cell density compositions and substantially lower shipping costs. Given the prospects for rapid advancement in the development of more effective and powerful microbial inoculants, consumers will benefit greatly from this ability to rapidly deliver microbe-based products.

### EXAMPLES

A greater understanding of the present invention and of its many advantages may be had from the following examples, given by way of illustration. The following examples are illustrative of some of the methods, applications, embodiments and variants of the present invention. They are not to be considered as limiting the invention. Numerous changes and modifications can be made with respect to the invention.

### Example 1—Exemplary Composition #1

Exemplified herein is a composition according to certain embodiments of subject invention for use in reducing GHGs, improving carbon utilization, and/or enhancing sequestration of carbon. This example is not to be intended as limiting. Formulations comprising other species of microorganisms, either in lieu of, or in addition to, those exemplified here, may be included in the composition.

The composition comprises a microbial inoculant comprising a Trichoderma spp. fungus and a Bacillus spp. bacterium. In specific instances, the composition comprises Trichoderma harzianum and Bacillus amyloliquefaciens. Even more specifically, the strain of B. amyloliquefaciens can be B. amyloliquefaciens NRRL B-67928.

In one embodiment, the composition can comprise from 1 to 99% Trichoderma by weight and from 99 to 1% Bacillus by weight. In some embodiments, the cell count ratio of Trichoderma to Bacillus is about 1:9 to about 9:1, about 1:8 to about 8:1, about 1:7 to about 7:1, about 1:6 to about 6:1, about 1:5 to about 5:1 or about 1:4 to about 4:1.

The composition can comprise about 1×10⁶ to 1×10¹², 1×10⁷ to 1×10¹¹, 1×10⁸ to 1×10¹⁰, or 1×10⁹ CFU/ml of the Trichoderma; and about 1×10⁶ to 1×10¹², 1×10⁷ to 1×10¹¹, 1×10⁸ to 1×10¹⁰, or 1×10⁹ CFU/ml of the Bacillus.

In specific instances, the composition comprises 10.0% by weight of the microbial inoculant, and 90% by weight water, where the inoculant comprises 1×10¹ CFU/mL Trichoderma harzianum and 1×10⁹ CFU/mL of Bacillus amyloliquefaciens.

### Example 2—Exemplary Composition #2

A sealable pouch can be used to store and transport a product comprising a product containing at least 1×10⁸ CFU/ml of Wickerhamomyces anomalus blended with residual microbial fermentation broth (e.g., 10.0% microbial inoculant and 90% broth by volume). In preferred embodiments, the strain is NRRL Y-68030. Other components can be added to the product, e.g., micronutrients, macronutrients, prebiotics and/or other microbes similarly produced.

The product is then diluted with water in a mixing tank to a concentration of 1×10⁶ to 1×10⁷ CFU/ml. One bag can be used to treat approximately 1-10 acres of crop or citrus grove.

### Example 3—Additional Components—Starter Materials

The microbial composition of Examples 1 and 2 can be mixed with and/or applied concurrently with additional “starter” materials to promote initial growth of the microorganisms in the composition. These can include, for example, prebiotics and/or nano-fertilizers (e.g., Aqua-Yield, NanoGrol™), as well as biochar (e.g., from 5% to 50% biochar to volume of soil, or 0.1-0.5 lb per square foot of soil).

An exemplary formulation of such growth-promoting “starter” materials comprises one or more of the following ingredients by weight, in any combination:
Soluble potash (K₂O) (1.0% to 2.5%, or about 2.0%)
Magnesium (Mg) (0.25% to 0.75%, or about 0.5%)
Sulfur (S) (2.5% to 3.0%, or about 2.7%)
Boron (B) (0.01% to 0.05%, or about 0.02%)
Iron (Fe) (0.25% to 0.75%, or about 0.5%)
Manganese (Mn) (0.25% to 0.75%, or about 0.5%)
Zinc (Zn) (0.25% to 0.75%, or about 0.5%)
Humic acid (8% to 12%, or about 10%)
Kelp extract (5% to 10%, or about 6%)
Water (70% to 85%, or about 77% to 80%)

The microbial inoculant, and/or optional growth-promoting “starter” materials, can be applied dry to soil and/or mixed with water in an irrigation system tank and applied to soil.

### Example 4—Microbial Strains

The subject invention utilizes beneficial microbial strains. In certain embodiments, the microorganism is a strain of Trichoderma, such as, e.g., a strain of T. harzianum, T viride, T. longibrachia, T. asperellwn, T. hamatum, T. koningii, T. reesei, T. guizhouse and/or others.

Exemplary Trichoderma harzianum strains can include, but are not limited to, T-315 (ATCC 20671); T-35 (ATCC 20691); 1295-7 (ATCC 20846); 1295-22 [T-22] (ATCC 20847); 1295-74 (ATCC 20848); 1295-106 (ATCC 20873); T12 (ATCC 56678); WT-6 (ATCC 52443): Rifa T-77 (CMI CC 333646); T-95 (60850); T12m (ATCC 20737); SK-55 (No. 13327; BP 4326 NIBH (Japan)); RR17Bc (ATCC PTA 9708); TSHTH20-1 (ATCC PTA 10317); AB 63-3 (ATCC 18647); OMZ 779 (ATCC 201359); WC 47695 (ATCC 201575); m5 (ATCC 201645); (ATCC 204065); UPM-29 (ATCC 204075); T-39 (EPA 119200); and/or F11Bab (ATCC PTA 9709).

In some embodiments, the microbe is a Bacillus strain, such as, e.g., B. subtilis, B. amyloliquefaciens, B. licheniformis, B. megaterium, B. polymyxa and/or others.

B. subtilis strains can include, e.g., B. subtilis strain B1 (ATCC PTA-123459) and/or strain B4 (NRRL B-68031).

Bacillus amyloliquefaciens strains can include, but are not limited to, NRRL B-67928, FZB24 (EPA 72098-5; BGSC 10A6), TA208, NJN-6, N2-4, N3-8, and those having ATCC accession numbers 23842, 23844, 23843, 23845, 23350 (strain DSM 7), 27505, 31592, 49763, 53495, 700385, BAA-390, PTA-7544, PTA-7545, PTA-7546, PTA-7549, PTA-7791, PTA-5819, PTA-7542, PTA-7790, and/or PTA-7541.

### Example 5—Corn Yield with Fertilizer Reduction

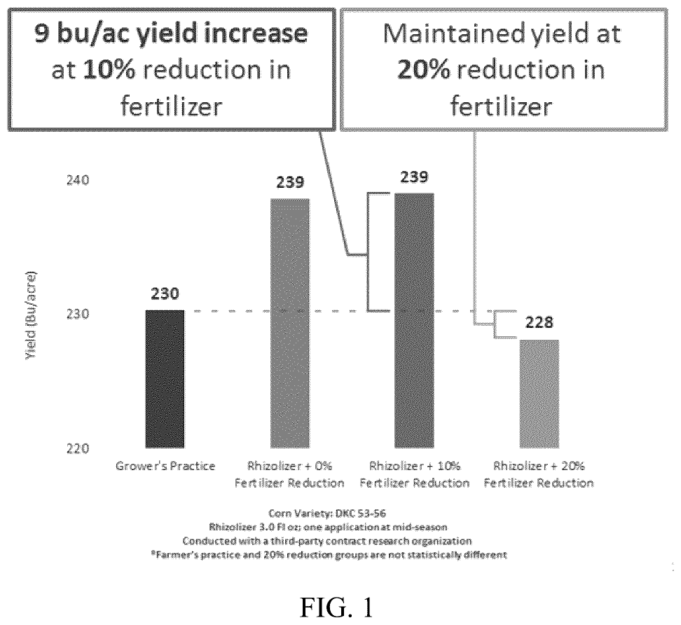

A composition according to Example 1 (“composition 1”) was applied at 3.0 fl oz/acre to a DKC 53-56 variety corn crop in Walworth County, WI, at mid-season, alongside varying concentrations of fertilizer and compared with grower's practice fertilization (control). A 9 bu/ac increase in yield was observed with a 10% reduction in fertilizer over grower's practice. FIG. 1. Yield was maintained compared to control when composition 1 was applied at 20% reduction in fertilizer usage.

### Example 6—Yield Increase Over Grower's Practice

### Corn Yield Increase

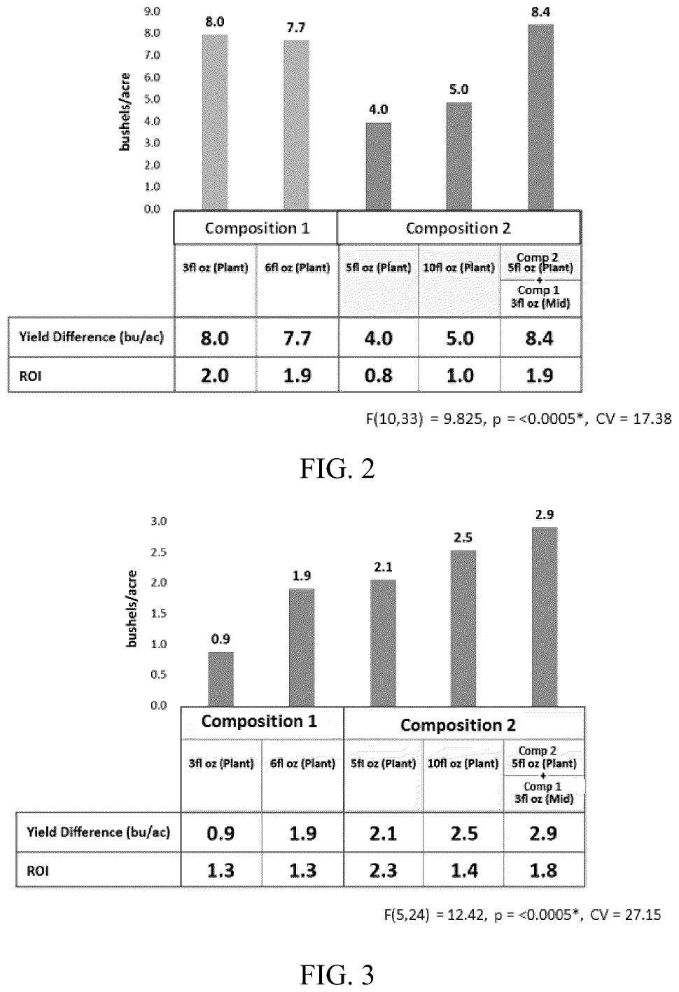

Composition 1 and a composition of Example 2 (“composition 2”) were applied according to the dosage and timing outlined in FIG. 2. The corn yield increase win rate of 34/41 paired comparisons was 83%. The highest average yield difference was observed when composition 2 was applied at planting and composition 1 was applied at mid-season.

### Soybean Yield Increase

Composition 1 and 2 were applied according to the dosage and timing outlined in FIG. 3. The soybean yield increase win rate of 23/30 paired comparisons was 77%. The highest average yield difference was observed when composition 2 was applied at planting and composition 1 was applied at mid-season.

Return of Investment assumptions were as follows:
Corn price: $5.00/Bu
Soybean price: $14.00/Bu
Composition 1 cost: $3.33/fl oz
Composition 2 cost: $2.50/fl oz

### Example 7—Carbon Sequestration

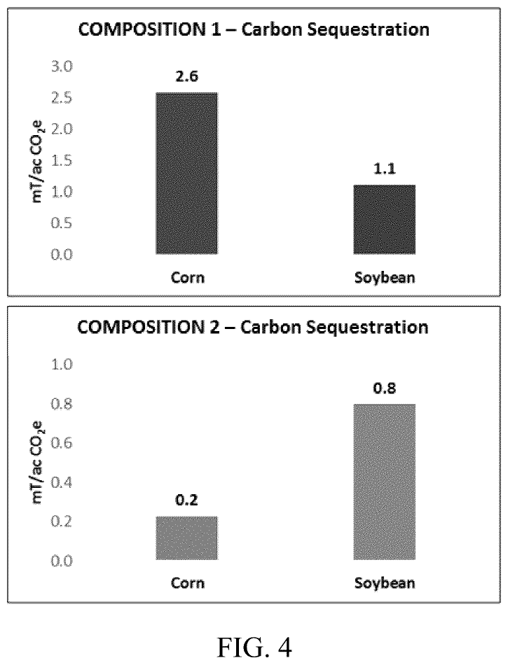

As shown in FIG. 4, composition 1 soil carbon (CO₂e mT/Ac) showed an increase over the Grower's Practice across all treatments: 2.6 mT/Ac for corn and 1.1 mT/Ac for soybean (2 and 4 comparisons, respectively).

Composition 2 soil carbon (CO₂e mT/ac) showed an increase over the Grower's Practice across all treatments: 0.2 mT/Ac for corn and 0.8 mT/ac for soybean (1 and 2 comparisons, respectively).

In corn, split applications for both Composition 1 and 2 sequestered more carbon than the single at-plant application.

### Example 8-Nitrous Oxide Reduction

In some embodiments, reduction in nitrous oxide emissions is an important aspect of the subject holistic systems. Agricultural soils are sources of nitrous oxide emissions due, in part, to nitrogen-rich fertilizer usage.

The reduction in nitrous oxide emissions as a result of application of composition 1 was studied in a corn field in South Dakota. The soil type was a Brandt silty clay loam. Corn (P9188AM) was planted in early June (Day 1) at a population of 32,000 seeds per acre.

Treatment 1 consisted of applying a urea-slurry at a rate of 150 kg-N/ha with 3 oz./acre of composition 1 and 6.4 oz./acre of a starter composition as described in Example 3 above (Day 3). A second application of composition 1 was applied at 3 oz./acre on Day 23. Treatment 2 consisted of the urea-slurry without composition 1. Treatment 3 consisted of untreated control.

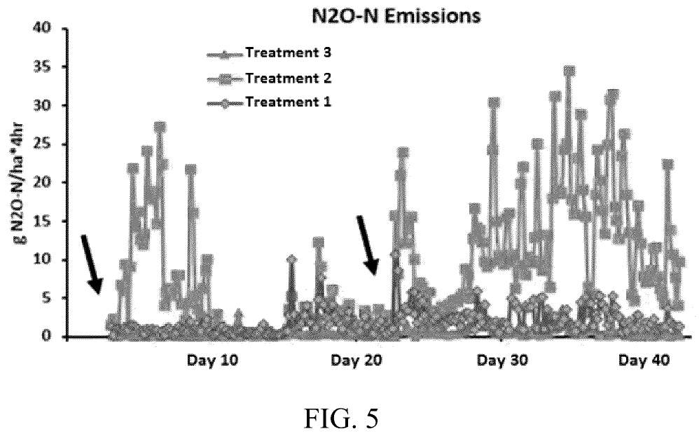

Fixed spectrophotometric chambers (LI-COR LI-8100-104 long-term opaque chambers (8100-104 LI-COR, Lincoln, NE) connected to a Picarro® Cavity Ringdown Spectrometer (model G2508; Picarro Inc., Santa Clara, CA) measured nitrous oxide emissions continuously and aggregated the data 6 times per day. FIG. 5.

Between Day 3 and Day 23, Treatment 1 resulted in average nitrous oxide emissions at 27% the level of Treatment 2. Between Day 24 and Day 43, Treatment 1 resulted in average nitrous oxide emissions at 12% the level of Treatment 2. After Day 43, minimal nitrous oxide emissions were measured.

### Example 9—Nutrient Uptake Study

A small plot study on DKC61-40 corn was conducted in Champaign, Illinois to compare an untreated standard grower's practice against various soil treatment compositions with regard to nutrient uptake. Each treatment was applied at planting over a 10×50 foot plot, and consisted of 4 replicates, for a total of 20 plots total. See Table 1.

TABLE 1
Treatment List

| In-Furrow | | Y-Drop Mid-Season | | |
|---|---|---|---|---|
| Treatment * | | Treatment | Rate | |
| 1 | Control | — | — | |
| 2 | Composition 2 | — | 10 oz/acre | |
| | Starter material | | 20 gram/acre | |
| 3 | Composition 2 | — | 5 oz/acre | |
| | Starter material | | 10 gram/acre | |
| 4 | Composition 1 | — | 6 oz/acre | |
| | Starter material | | 12.8 oz/acre | |
| 5 | Composition 2 | Composition 1 | 5 oz/acre | 3 oz/acre |
| | Starter material | Starter material | 10 gram/acre | 6.4 oz/acre |

* All plots received a base rate 10-34-0 (liquid ammonium phosphate) at 16 lb P₂O₅/acre in-furrow and 180 lb N/acre applied as UAN (32-0-0) (urea ammonium nitrate) pre-plant broadcast.

As shown in Table 2, Treatment 2 exhibited an increase in R1 plant nitrogen concentration over the control.

TABLE 2
Influence of Treatments on R1 Plant N, P and K Concentrations

| | N | P | K |
|---|---|---|---|
| Treatment | % | % | % |
| 1 | 2.07 | 0.292 | 1.23 |
| 2 | 1.94 | 0.282 | 1.16 |
| 3 | 2.11 | 0.307 | 1.28 |
| 4 | 2.07 | 0.302 | 1.23 |
| 5 | 2.14 | 0.305 | 1.30 |

As shown in Table 3, Treatment 3 and Treatment 5 exhibited an increase in R1 plant tissue P₂O₅ and K₂O uptake.

TABLE 3
Influence of Treatments on R1 Plant
N, P₂O₅ and K₂O Accumulation

| | N | P₂O₅ | K₂O |
|---|---|---|---|
| Treatment | lb/acre | lb/acre | lb/acre |
| 1 | 127.2 | 38.7 | 84.7 |
| 2 | 116.8 | 38.7 | 84.2 |
| 3 | 133.8 | 44.3 | 98.7 |
| 4 | 129.0 | 42.8 | 91.9 |
| 5 | 134.4 | 43.9 | 98.4 |

Additionally, as shown in Table 4, Treatment 5 also exhibited increased accumulation of S, Zn, B and Mn over the control.

TABLE 4
Influence of Treatments on R1 Plant
S, Zn, B and Mn Accumulation

| | S | Zr | B | Mn |
|---|---|---|---|---|
| Treatment | lb/acre | oz/acre | oz/acre | oz/acre |
| 1 | 8.86 | 3.14 | 0.917 | 2.77 |
| 2 | 8.05 | 3.12 | 0.820 | 2.86 |
| 3 | 8.75 | 3.16 | 0.984 | 2.88 |
| 4 | 8.69 | 3.18 | 0.913 | 2.85 |
| 5 | 9.14 | 3.24 | 0.971 | 3.19 |

### REFERENCES

Brummell, M. E., and S. D. Siciliano. (2011). “Measurement of Carbon Dioxide, Methane, Nitrous Oxide, and Water Potential in Soil Ecosystems.” Methods in Enzymology. 496:115-137. Doi: 10.1016/B978-0-12-386489-5.00005-1. (“Brummell and Siciliano 2011”).
Grandy, A. S. and G. P. Robertson (2007). “Land-Use Intensity Effects on Soil Organic Carbon Accumulation Rates and Mechanisms.” Ecosystems 10:58-73. (“Grandy 2007”).
Kallenbach, C. M. et al. (2015). “Microbial physiology and necromass regulate agricultural soil carbon accumulation.” Soil Biol & Biochem 91:279-290. (“Kallenbach 2015”).
Kallenbach, C. M. et al. (2019). “Managing Agroecosystems for Soil Microbial Carbon Use Efficiency: Ecological Unknowns, Potential Outcomes, and a Path Forward.” Frontiers in Microbiol 10:1146. (“Kallenbach 2019”).
Panettieri, M. et al. (2013). “Moldboard plowing effects on soil aggregation and soil organic matter quality assessed by 13C CPMAS NMR and biochemical analyses.” Agric., Ecosys & Envt 177:48-57. (“Panettieri 2013”).
Possinger, A. R. et al. (2020). “Organo-organic and organo-mineral interfaces in soil at the nanometer scale.” Nature comm. 11:6103. (“Possinger 2020”).
Soil Survey Staff, USDA (2014). “Keys to Soil Taxonomy.” USDA Natural Resources Conservation Service. 12^{th} Edition. (“USDA 2014”).
Trivedi, P. et al. (2015). “Soil aggregate size mediates the impacts of cropping regimes on soil carbon and microbial communities.” Soil Biol & Biochem 91:169-181. (“Trivedi 2015”).
Trivedi, P. et al. (2017). “Soil aggregation and associated microbial communities modify the impact of agricultural management on carbon content.” Envtl Microbiol 19(8), 3070-3086. (“Trivedi 2017”).
Warncke, D. D., (2014). “Managing Muck Soils for Vegetable Crops.” Soil Fertility and Plant Nutrition, Michigan State University. (“Warncke 2014”). http://www.hort.cornell.edu/expo/proceedings/2014/Cover%20Crops%20Tillage%20and%20 Soils/Muck%20Soils%20Warncke.pdf

1. A method for:
1) Enhancing soil health though rebuilding of degraded soils, preventing degradation of soils, and/or improving soil aggregate formation;
2) Enhancing plant health, growth and/or yields, including livestock feed crops;
3) Reducing chemical or petroleum-based surfactant usage;
4) Improving dispersion of water, nutrients and salts in soil;
5) Reducing fertilizer usage;
6) Recharging aquifers;
7) Improving crop and livestock water use efficiency;
8) Reducing atmospheric greenhouse gas emissions resulting from agricultural practices and livestock feed production, including manure management;
9) Enhancing sequestration of carbon in soil, vegetation and microbial biomass; and/or
10) Reducing the carbon footprint and/or carbon intensity of producing crops and livestock, which can also result in a reduction in the number of carbon credits used by a farmer or livestock producer,
wherein the method comprises applying a soil treatment composition comprising one or more soil-colonizing microorganisms, and/or a growth by-product thereof, to the plant and/or its surrounding environment, wherein the one or more microorganisms are selected from Bacillus spp., Trichoderma spp., Pleurotus spp., Saccharomyces spp., Debaryomyces spp., Lentinula spp., Wickerhamomyces spp., Starmerella spp., Meyerozyma spp., Pseudomonas spp., Pichia spp., Azotobacter spp., Azospirillum spp., Klebsiella spp., Frateuria spp., Myxococcus spp., and mycorrhizal fungi.

2. The method of claim 1, wherein the soil treatment composition comprises fermentation medium in which the one or more microorganisms were cultivated.

3. The method of claim 1, wherein the soil treatment composition comprises Trichoderma harzianum.

4. The method of claim 1, wherein the soil treatment composition comprises Bacillus amyloliquefaciens.

5. The method of claim 1, wherein the soil treatment composition comprises Trichoderma harzianum and Bacillus amyloliquefaciens NRRL B-67928.

6. The method of claim 5, wherein the soil treatment composition comprises a cell count ratio of 1:4, Trichoderma to Bacillus.

7. The method of claim 1, wherein the soil treatment composition comprises Wickerhamomyces anomalus NRRL Y-68030.

8. The method of claim 1, wherein the soil treatment composition comprises Bacillus subtilis B4 NRRL B-68031.

9. (canceled)

10. The method of claim 1, wherein the soil treatment composition comprises the microbial growth by-product without active microbial cells.

11. The method of claim 10, wherein the microbial growth by-product is a biosurfactant, and wherein the biosurfactant is in crude form in a fermentation medium in which it was produced.

12. The method of claim 10, wherein the microbial growth by-product is a sophorolipid biosurfactant.

13. The method of claim 1, wherein the soil treatment composition is applied in the form of dry powder or dry granules.

14. The method of claim 1, wherein the one or more microorganisms and/or growth by-products thereof are contacted directly with the plant's roots.

15. (canceled)

16. The method of claim 1, wherein one or more microorganisms and/or growth by-products thereof are mixed with water prior to application.

17. The method of claim 1, wherein the one or more microorganisms and/or growth by-products thereof are applied to a plant and/or to soil using an irrigation system.

18. The method of claim 1, wherein the one or more microorganisms and/or growth by-products thereof are applied to the plant and/or its surrounding environment contemporaneously with a source of one or more nutrients selected from nitrogen, phosphorous, and potassium.

19. The method of claim 1, wherein the one or more microorganisms and/or growth by-products thereof are applied to the plant and/or its surrounding environment contemporaneously with prebiotics selected from kelp extract, fulvic acid, chitin, biochar, humate and humic acid.

20. The method of claim 1, wherein the one or more microorganisms and/or growth by-products thereof are applied to the plant and/or its surrounding environment contemporaneously with rock dust.

21. The method of claim 1, wherein the plant is provided to a livestock animal such that the animal consumes the plant.

22. The method of claim 1, further comprising monitoring one or more of soil moisture content, soil microbial populations, water table levels, nutrient content, and GHG emissions at the location of application.
